(19) United States
(12) Patent Application Publication
(10) Pub. No.: US 2010/0285995 A1
(43) Pub. Date: Nov. 11, 2010

(54) Identification and Characterization of Pregnancy-Associated Genetic Signatures and Use Thereof for Diagnosis and Treatment of Breast Cancer

(76) Inventors: Jose Russo, Rydal, PA (US); Irma Russo, Rydal, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/811,303

(22) PCT Filed: Jan. 2, 2009
(86) PCT No.: PCT/US09/30031

§ 371 Date: Jun. 30, 2010

Related U.S. Application Data
(60) Provisional application No. 61/018,570, filed on Jan. 2, 2008.

(51) Int. Cl. C40B 40/06; C07H 21/04; C07K 14/435; G01N 33/574; C07K 16/18; C07H 21/02; C12Q 1/68

(52) U.S. Cl. 506/16; 536/23.1; 530/350; 435/7.23; 530/387.1; 536/24.5; 435/6

(57) ABSTRACT

Compositions and methods for the diagnosis and treatment of breast cancer are provided.

Pursuant to 35 U.S.C. §202(c), it is acknowledged that the U.S. Government has certain rights in the invention described, which was made in part with funds from the National Institutes of Health, Grant Number CA093599.

### FIELD OF THE INVENTION

This invention relates to the fields of molecular biology, genetics and breast cancer. More specifically, the invention provides a genetic signature associated with reduced risk of breast cancer. Methods and kits for using the sequences so identified for diagnostic and therapeutic treatment purposes are also provided, as are therapeutic compositions for treatment of breast cancer.

### BACKGROUND OF THE INVENTION

Several publications and patent documents are cited throughout the specification in order to describe the state of the art to which this invention pertains. Each of these citations is incorporated herein by reference as though set forth in full.

More than 300 years elapsed since a striking excess in breast cancer mortality was reported in nuns, in whom the increased risk was attributed to their childlessness [1] until MacMahon et al. [2], in a landmark case-control study found an almost linear relationship between a woman's risk and the age at which she bore her first child. This work, that included areas of high, intermediate and low breast cancer risk in seven parts of the world, confirmed that pregnancy had a protective effect that was evident from the early teen years and persisted until the middle twenties [2]. Other studies have reported that additional pregnancies and breastfeeding confer greater protection to young women, including a statistically significantly reduced risk of breast cancer in women with deleterious BRCA1 mutations who breast-fed for a cumulative total of more than 1 year [3,4]. Our studies, designed for unraveling what specific phenomena occurred in the breast during pregnancy for conferring a lifetime protection from developing cancer, led us to the discovery that endogenous endocrinological or environmental influences affecting breast development before the first full term pregnancy were important modulators of the susceptibility of the breast to undergo neoplastic transformation [5,6]. The fact that exposure of the immature breast of young nulliparous females to environmental physical agents [7] or chemical toxicants [8, 9] results in a greater rate of cell transformation has been demonstrated through the identification of the cell of origin of chemically induced mammary cancer in rodents [10] and the stem cell of breast cancer in women's breast, and confirmed by studies in an in vitro model [5, 6].

The protection conferred by pregnancy, however, is age-specific, since a delay in childbearing after age 24 progressively increases the risk of cancer development, which becomes greater than that of nulliparous women when the first full term pregnancy (FFTP) occurs after 35 years of age [2,11]. The higher breast cancer risk that has been associated with early menarche [12] further emphasizes the importance of the length of the susceptibility “window” that encompasses the period of breast development occurring between menarche and the first pregnancy, when the organ is more susceptible to either undergo complete differentiation under physiological hormonal stimuli, and hence to be protected from breast cancer, or to suffer genetic or epigenetic damage that might contribute to increasing the lifetime risk of developing breast cancer [9,13].

The damage caused by a single or a combination of putative cancer causing agents might, in turn, be amplified by the genetic make up of the patient, such as the inheritance of the BRCA1 or BRCA2 susceptibility genes, which influences the pattern of breast development and differentiation and is responsible for at least 5% of all the breast cancer cases [14-16]. This postulate is supported by our observations that the architectural pattern of lobular development in parous women with cancer differs from that of parous women without cancer, being similar to that of nulliparous women with or without cancer. Thus, the higher breast cancer risk in parous women might have resulted from either a failure of the breast to fully differentiate under the influence of the hormones of pregnancy [17,18] and/or stimulation of the growth of foci of transformed cells initiated by early damage or genetic predisposition [9,13,15].

### SUMMARY OF THE INVENTION

In accordance with the present invention, we have performed genetic profiling analyses to identify genes associated with the protective effects conferred by pregnancy on the development of breast cancer. These genes are listed in the tables presented herein. We have identified a specific genomic profile that is still identifiable in parous women at post-menopause. Our data also reveal that this genomic signature is constituted by genes that cluster differently than those genes expressed in the epithelial cells of parous and non-parous women with breast cancer as well as from nulliparous women without breast cancer. This genomic signature has enabled us to evaluate the degree of mammary gland differentiation induced by pregnancy and identify the genetic signature associated with development of the beneficial Stem Cell 2 phenotype. Moreover, further characterization of the fully differentiated condition of the breast epithelium that is associated with reduced cancer risk and the genetic signature associated with this condition, provides a useful molecular tool for identifying those patients in which pregnancy has been protective, and for identifying women at risk irrespective of their pregnancy history. In a preferred embodiment, the differentially expressed nucleic acids are provided in Tables 2, 3 or 4, said differential expression being associated with a reduced risk of breast cancer conferred by full term pregnancy, said signature comprising at least 4, 5 or 10 of the differentially expressed nucleic acids in the aforementioned tables. A plurality of protein products encoded by the nucleic acids set forth in Table 2, 3 or 4 are also provided in the present invention. Such protein products provide new targets for use in screening assays to identify therapeutic agents useful for the treatment of breast cancer.

Thus, in yet another aspect of the invention, methods are provided for identifying agents which modulate the activity of differentially regulated genes that are involved in cancer progression in the breast. The invention also encompasses agents identified using the aforementioned methods and methods of use of such agents alone and in combination for the treatment of breast cancer.

In yet another embodiment, a method for diagnosing a reduced risk for the development of breast cancer in a patient is also disclosed. An exemplary method comprises obtaining a sample of breast cells from said patient; determining differential expression levels of nucleic acids isolated from said cells thereby obtaining a genetic signature from said patient; and comparing the genetic signature from said patient to the genetic signatures of provided in Tables 2, 3 or 4, wherein when said signatures are comparable, said patient has a reduced risk for developing breast cancer.

Finally, kits for practicing the method described above are also encompassed by the present invention.

### BRIEF DESCRIPTION OF THE DRAWINGS

FIG. 1: Unsupervised hierarchical clustering analysis performed using the expression profiles of 2,541 globally varying genes across the nulliparous and parous data sets representing red lines—Parous controls, green lines—Parous cases, blue lines-Nulliparous controls, and yellow lines—Nulliparous cases. The clustering procedure used to derive the dendrogram is described in the Methods section.

FIG. 2, (a) and (b): Unsupervised hierarchical analysis of subsets of 18 matched breast epithelia from the parous control specimens shown in FIG. 1 that were microdissected and hybridized independently as biological replicates. The combined parity/absence of breast cancer data generated a distinct genomic profile that differed from those of the breast cancer groups, irrespective of parity history, and from the nulliparous cancer free group. Groups identified as for FIG. 1.

### DETAILED DESCRIPTION OF THE INVENTION

Breast cancer risk has traditionally been linked to nulliparity or late first full term pregnancy, whereas young age at first childbirth, multiparity, and breast-feeding are associated with a reduced risk. Early pregnancy confers protection by inducing breast differentiation, which imprints a specific and permanent genomic signature in experimental rodent models. For testing whether the same phenomenon was detectable in the atrophic breast of postmenopausal parous women, we designed a case-control study for the analysis of the gene expression profile of RNA extracted from epithelial cells microdissected from normal breast tissues obtained from 18 parous and 7 nulliparous women free of breast pathology (controls), and 41 parous and 8 nulliparous women with history of breast cancer (cases). RNA was hybridized to cDNA glassmicroarrays containing 40,000 genes; arrays were scanned and the images were analyzed using ImaGene software version 4,2. Normalization and statistical analysis were carried out using LIMMA and GeneSight software was used for hierarchical clustering. The parous control group contained 2,541 gene sequences representing 18 biological processes that were differentially expressed in comparison with the other three groups. Hierarchical clustering of these genes revealed that the combined parity/absence of breast cancer data generated a distinct genomic profile that differs from those of the breast cancer groups, irrespective of parity history, and from the nulliparous cancer free group, which has been traditionally identified as a high risk group.

The signature that identifies those women in whom parity has been protective will serve as a molecular biomarker of differentiation for evaluating the potential use of preventive agents.

The following definitions are provided to facilitate an understanding of the present invention.

The phrase “genetic signature” refers to a plurality of nucleic acid molecules whose expression levels are indicative of a given metabolic or pathological state. The genetic signatures described herein can be employed to characterize at the molecular level the fully differentiated condition of the breast epithelium that is associated with a reduction in breast cancer risk, thus providing a useful molecular tool for predicting when pregnancy has been protective, for identifying women at risk irrespective of their pregnancy history, and for use as an intermediate biomarker in assays for evaluating cancer preventive agents.

For purposes of the present invention, “a” or “an” entity refers to one or more of that entity; for example, “a cDNA” refers to one or more cDNA or at least one cDNA. The terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose. “Sample” or “patient sample” or “biological sample” generally refers to a sample which may be tested for a particular molecule, preferably a genetic signature specific marker molecule, such as a marker shown in the tables provided below. Samples may include but are not limited to cells, body fluids, including blood, serum, plasma, urine, saliva, tears, pleural fluid and the like.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any specific marker gene or nucleic acid, but does not hybridize to other human nucleotides. Also polynucleotide which “specifically hybridizes” may hybridize only to a specific marker, such a genetic signature-specific marker shown in Tables 2, 3 and 4. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63 (% formamide)−600/# bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide” or “oligo” as used herein means a short sequence of DNA or DNA derivatives typically 8 to 35 nucleotides in length, primers, or probes. An oligonucleotide can be derived synthetically, by cloning or by amplification. An oligo is defined as a nucleic acid molecule comprised of two or more ribo- or deoxyribonucleotides, preferably more than three. The exact size of the oligonucleotide will depend on various factors and on the particular application and use of the oligonucleotide. The term “derivative” is intended to include any of the above described variants when comprising an additional chemical moiety not normally a part of these molecules. These chemical moieties can have varying purposes including, improving solubility, absorption, biological half life, decreasing toxicity and eliminating or decreasing undesirable side effects.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting elevated mRNA levels associated with breast cancer may be between 15-35 nucleotides in length, and more typically about 21 nucleotides in length.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the breast cancer specific marker nucleic acid molecule(s) such that the latter is transcribed into mRNA. Host cell machinery then translates mRNA into a polypeptide.

Those skilled in the art will recognize that a nucleic acid vector can contain nucleic acid elements other than the promoter element and the breast cancer protective specific marker gene nucleic acid molecule(s). These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated genetic signature nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule or combination of molecules, preferably a combination of the genetic signature marker molecules, such as a combination of the markers shown in Tables 2, 3 and 4. Samples may include but are not limited to cells, body fluids, including blood, serum, plasma, nipple aspirates, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid based molecule, for example an oligo, which exhibits the capacity to modulate the activity of the genetic signature nucleic acids described herein or their encoded proteins. Agents are evaluated for potential biological activity by inclusion in screening assays described herein below.

The term “modulate” as used herein refers increasing or decreasing. For example, the term modulate refers to the ability of a compound or test agent to either interfere with, or augment signaling or activity of a gene or protein of the present invention.

### Methods of Using the Genetic Signatures of the Invention

Genetic signature containing nucleic acids, including but not limited to those listed in Tables 2, 3, and 4 may be used for a variety of purposes in accordance with the present invention. The genetic signature associated with a reduction in breast cancer risk (e.g., the plurality of nucleic acids contained therein) containing DNA, RNA, or fragments thereof may be used as probes to detect the presence of and/or expression of these specific markers in a biological sample. Methods in which such marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting the genetic signature may be conducted on any type of biological sample, but is most preferably performed on breast tissue. From the foregoing discussion, it can be seen that genetic signature containing nucleic acids, vectors expressing the same, genetic signature encoded proteins and anti-genetic signature encoded protein specific antibodies of the invention can be used to detect the signature in body tissue, cells, or fluid, and alter genetic signature containing marker protein expression for purposes of assessing the genetic and protein interactions involved in breast cancer.

In most embodiments for screening for genetic signature containing nucleic acid(s), the sample will initially be amplified, e.g. using PCR, to increase the amount of the template as compared to other sequences present in the sample. This allows the target sequences to be detected with a high degree of sensitivity if they are present in the sample. This initial step may be avoided by using highly sensitive array techniques that are becoming increasingly important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus, any of the aforementioned techniques may be used to detect or quantify genetic signature expression and accordingly, diagnose patient susceptibility for developing breast cancer.

### Kits and Articles of Manufacture

Any of the aforementioned products can be incorporated into a kit which may contain genetic signature polynucleotides or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

### Methods of Using the Genetic Signature for Development of Therapeutic Agents

Since the genetic signature identified herein has been associated with the etiology of breast cancer, methods for identifying agents that modulate the activity of the genes and their encoded products should result in the generation of efficacious therapeutic agents for the treatment of a cancer, particularly breast cancer.

The nucleic acids comprising the signature contain regions which provide suitable targets for the rational design of therapeutic agents which modulate their activity. Small peptide molecules corresponding to these regions may be used to advantage in the design of therapeutic agents which effectively modulate the activity of the encoded proteins. Molecular modeling should facilitate the identification of specific organic molecules with capacity to bind to the active site of the proteins encoded by the genetic signature nucleic acids based on conformation or key amino acid residues required for function. A combinatorial chemistry approach will be used to identify molecules with greatest activity and then iterations of these molecules will be developed for further cycles of screening. In certain embodiments, candidate agents can be screening from large libraries of synthetic or natural compounds. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated in to pharmaceutical compositions and utilized for the treatment of breast cancer.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered breast cancer associated gene. These host cell lines or cells are defective at the polypeptide level. The host cell lines or cells are grown in the presence of drug compound. The effect on cellular morphology and/or proliferation of the host cells is measured to determine if the compound is capable of regulating the same in the defective cells. Host cells contemplated for use in the present invention include but are not limited to bacterial cells, fungal cells, insect cells, mammalian cells, particularly breast cells. The genetic signature encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

Breast cells and breast cell lines suitable for studying the effects of genetic signature expression on cellular morphology and signaling methods of use thereof for drug discovery are provided. Such cells and cell lines will be transfected with genetic signature encoding nucleic acids described herein and the effects on breast cell functions and/or breast cell apoptosis can be determined. Such cells and cell lines can also be contacted with the siRNA molecules provided herein to assess the effects thereof on malignant transformation. The siRNA molecules will be tested alone and in combination of 2, 3, 4, and 5 siRNAs to identify the most efficacious combination for down regulating target nucleic acids.

A wide variety of expression vectors are available that can be modified to express the novel DNA or RNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&H is (Invitrogen); baculovirus vectors such as pVL1392, pVL1393, or pAC360 (Invitrogen); and yeast vectors such as YRP17, YIP5, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the genetic signature of the present invention or functional fragments thereof provide a system in which to screen potential compounds or agents for the ability to modulate the development of breast cancer

Another approach entails the use of phage display libraries engineered to express fragment of the polypeptides encoded by the genetic signature containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacophore.

Thus, one may design drugs which have, e.g., improved polypeptide activity or stability or which act as inhibitors, agonists, antagonists, etc. of polypeptide activity. By virtue of the availability of the genetic signature containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of genetic signature containing nucleic acids enables the production of strains of laboratory mice carrying the signature of the invention. Transgenic mice expressing the genetic signature of the invention provide a model system in which to examine the role of the protein(s) encoded by the signature containing nucleic acid in the development and progression towards breast cancer. Methods of introducing transgenes in laboratory mice are known to those of skill in the art. Three common methods include: (1) integration of retroviral vectors encoding the foreign gene of interest into an early embryo; (2) injection of DNA into the pronucleus of a newly fertilized egg; and (3) the incorporation of genetically manipulated embryonic stem cells into an early embryo. Production of the transgenic mice described above will facilitate the molecular elucidation of the role that a target protein plays in various cellular metabolic processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extra-chromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of genetic signature containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use genetic signature associated genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extra-chromosomal recombination, which occurs at a frequency approaching 100%, homologous plasmid-chromosome recombination was originally reported to only be detected at frequencies between 10⁻⁶ and 10⁻³. Non-homologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing genetic signature containing nucleic acid as a targeted insertional cassette provides means to detect a successful insertion as visualized, for example, by acquisition of immunoreactivity to an antibody immunologically specific for the polypeptide encoded genetic signature nucleic acid(s) and, therefore, facilitates screening/selection of ES cells with the desired genotype.

As used herein, a knock-in animal is one in which the endogenous murine gene, for example, has been replaced with human genetic signature-associated gene(s) of the invention. Such knock-in animals provide an ideal model system for studying the development of breast cancer.

As used herein, the expression of a genetic signature containing nucleic acid, fragment thereof, or genetic signature fusion protein can be targeted in a “tissue specific manner” or “cell type specific manner” using a vector in which nucleic acid sequences encoding all or a portion of genetic signature-associated protein are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific expression of proteins are well known in the art and described herein.

Methods of use for the transgenic mice of the invention are also provided herein. Transgenic mice into which a nucleic acid containing the genetic signature or its encoded protein(s) have been introduced are useful, for example, to develop screening methods to screen therapeutic agents to identify those capable of modulating the development of breast cancer.

### Pharmaceuticals and Peptide Therapies

The elucidation of the role played by the gene products described herein in breast cancer progression facilitates the development of pharmaceutical compositions useful for treatment and diagnosis of breast cancer. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As it is presently understood, RNA interference involves a multi-step process. Double stranded RNAs are cleaved by the endonuclease Dicer to generate nucleotide fragments (siRNA). The siRNA duplex is resolved into 2 single stranded RNAs, one strand being incorporated into a protein-containing complex where it functions as guide RNA to direct cleavage of the target RNA (Schwarz et al, Mol. Cell. 10:537 548 (2002), Zamore et al, Cell 101:25 33 (2000)), thus silencing a specific genetic message (see also Zeng et al, Proc. Natl. Acad. Sci. 100:9779 (2003)).

Pharmaceutical compositions that are useful in the methods of the invention may be administered systemically in parenteral, oral solid and liquid formulations, ophthalmic, suppository, aerosol, topical or other similar formulations. These pharmaceutical compositions may contain pharmaceutically-acceptable carriers and other ingredients known to enhance and facilitate drug administration. Thus such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate agent to a patient according to the methods of the invention. The use of nanoparticles to deliver agents, as well as cell membrane permeable peptide carriers that can be used are described in Crombez et al., Biochemical Society Transactions v35:p44 (2007).

In order to treat an individual having breast cancer, to alleviate a sign or symptom of the disease, the pharmaceutical agents of the invention should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of agent required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having breast cancer.

In an individual suffering from breast cancer, in particular a more severe form of the disease, administration of agent can be particularly useful when administered in combination, for example, with a conventional agent for treating such a disease. The skilled artisan would administer the agent alone or in combination and would monitor the effectiveness of such treatment using routine methods such as mammography, radiologic, immunologic or, where indicated, histopathologic methods. Other conventional agents for the treatment of breast cancer include anti cancer agents, such as herceptin and tamoxifen. Administration of the pharmaceutical preparation is preferably in an “effective amount” this being sufficient to show benefit to the individual. This amount prevents, alleviates, abates, or otherwise reduces the severity of breast cancer symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

The following examples are provided to illustrate certain embodiments of the invention. They are not intended to limit the invention in any way.

### Example 1

In order to understand how the dramatic modifications that occur during pregnancy in the pattern of lobular development and differentiation [17, 18], cell proliferation and steroid hormone receptor content of the breast [19] influence the cancer risk even after their regression after weaning and further after menopause, we have analyzed the pattern of gene expression occurring during and after pregnancy in rodents [20, 21]. Hierarchical cluster analysis of the genomic profile of rat mammary glands in the 15th and 21st days pregnancy and at 21 and 42 days post partum revealed four different patterns of expression in relation to the time of pregnancy [21]. During pregnancy, genes related to the secretory properties of the mammary epithelium (Cluster A) become upregulated, decreasing to control values after 21 and 42 days post-partum. Cluster B includes genes related to the apoptotic pathways, the fatty acid binding protein and cathecol-O-methyltransferase, among others, which become upregulated from the end of pregnancy until the 21st day postpartum, decreasing thereafter. Cluster C represents differentiation-associated genes whose level of expression continuously and progressively increases with time of pregnancy, reaching their highest levels between 21 and 42 days post-partum, and Cluster D comprises genes upregulated around the 15th day of pregnancy and become progressively down-regulated from the end of pregnancy until the 42nd day post-partum. These observations confirm at genomic level our previous morphological and physiological findings that indicate that temporal and sequential changes have to occur in the development of the mammary gland for accomplishing a protective degree of differentiation [20, 21,24]. The importance of identifying a specific signatures by 42 days post-partum is highlighted by the observations that administration of the polycyclic hydrocarbon 7,12-dimethylbenz(a)anthracene (DMBA) [22] or the alkylating agent N-methyl-N-nitrosourea (NMU) [23] to parous rats results in a markedly reduced tumorigenic response, supporting the concept that the differentiation induced by pregnancy results in a shift of the susceptible Stem Cell 1 to the refractory Stem Cell 2 [5,6].

The universality of this phenomenon has been further confirmed in various strains of rats and mice [24] and using different platforms for global genome analysis [21,25,26]. Studies in experimental animal models have been useful for uncovering the sequential genomic changes occurring in the mammary gland in response to the multiple hormonal stimuli of pregnancy that lead to the imprinting of a permanent genomic signature. Work reported here was designed with the purpose of testing whether a similar phenomenon occurs in the atrophic breast of postmenopausal parous women, specifically in the epithelium of lobules type 1 (Lob 1), the site of origin of breast carcinomas [5,6]. Our results support our hypothesis that parous women after menopause who had not developed breast cancer exhibit a genomic “signature” that differs from that present in the breast of parous postmenopausal women with cancer or in nulliparous women, who traditionally represent a high breast cancer risk group [1-6].

The following materials and methods are provided to facilitate the practice of Example I.

### Patients and Methods for Sample Collection

For this three-center hospital-based study patients were enrolled from the American Oncologic Hospital of the Fox Chase Cancer Center in Philadelphia, Pa., Christiana Care Health System, Newark, Del., and Somerset Medical Center, Sommerville, N.J. The study protocol had been approved by the Institutional Review Board of each participating institution, and written informed consent was obtained from every participant. Patients were eligible for the study if they met the following criteria: Postmenopausal women that were 50 years old or greater and whose menses had naturally ceased one year before enrollment. Excluded from this study were women whose ovaries had been surgically removed, who had a history of cancer other than non-melanoma skin cancer, who were taking medications that could interfere with the study protocol such as estrogens (including Tamoxifen and Raloxifene); progestins, androgens, prednisone, thyroid hormones, insulin), and women with Alzheimer's disease or severe cognitive deficit and were unable to give informed consent.

### Participant Identification:

Potential participants were identified by a trained research nurse that performed daily searches of surgical breast consultation visit summaries at the Breast Evaluation Clinic of the three participating hospitals. Those women that fulfilled the eligibility criteria listed above and that their treating breast surgeon recommended a breast biopsy were selected for the study. Information included in visit summaries such as age, menopausal status, history of cancer, and current medications were used to determine if a woman was potentially eligible for this study. A letter was sent to each potential participant describing the study and informing them of their eligibility, which was confirmed in a telephone interview placed within two weeks of initial clinical evaluations when biopsies were recommended.

### Data and Specimen Collection:

Data were collected at pre-operative clinic visits before biopsies and during breast biopsy procedures. At the pre-operative visits, informed consent was obtained, participants were asked to complete a study questionnaire and height and weight were measured. Each one of the participating hospitals was provided specifically designed kits for breast tissue collection that included tissue specimen containers partially filled with 70% ethanol, blood collecting tubes, copies of the eligibility criteria, patient data questionnaires, and labels with coded numbers for the biospecimens and questionnaires. All patients were accessed to a FCCC database using the originally assigned coded numbers. Patient names and medical record numbers were known only by the treating physician and authorized personnel at each participating hospitals. Breast tissue specimens were obtained by the operating surgeon following standard procedures for surgical breast biopsies at each site only after tissues were evaluated for presence of tumor, and if present, assessment of tumor size, margin identification, and adequacy of the tissue available for pathological diagnosis. Normal appearing tissues were taken from areas at a distance equal or greater than 2 cm from any grossly identifiable lesion and immediately fixed in 70% ethanol for eight-hours, followed by dehydration, paraffin embedding, sectioning and staining for histological analysis and laser capture microdissection (LCM) following procedures previously described [27]. Histopathological diagnosis of tumor type was made by Pathologists at each site. Only women diagnosed with invasive breast cancer (cases) or benign breast disease without hyperplasia or atypia (controls) were included in the study. From the 74 postmenopausal women that fulfilled the criteria of eligibility for this study, there were 59 (80%) parous and 15 (20%) nulliparous. Eighteen of the parous women that had benign breast biopsies but were free of cancer served as controls. At the time of biopsy they ranged in age from 50 to 78 years old (mean 63.23±8.78), and had had their first full term pregnancy between 17 and 34 years of age (mean age 24.23±4.93). Forty one women that had a diagnosis of breast cancer ranged in age from were selected as cases. The mean age at the time of breast cancer diagnosis was 69.35±9.21 and the first pregnancy occurred at 24.97±4.06 years of age. Among the nulliparous women, 7 were free of cancer (controls), and 8 had breast cancer (cases) in whom biopsies were performed when they had an average of 56.71±5.65 and 66.0±12.43 years of age, respectively (Table 1). The number of cases per group represents the distribution of cases at each one of the participating hospitals.

TABLE 1
Profile of the Four Groups of Patients* and Diagnosis of
Breast Lesions from which Normal Lobule type 1 Epithelium was
Obtained by Laser Capture Microdissection (LCM)

| | | | Age at | | | | | |
|---|---|---|---|---|---|---|---|---|
| | | Age at | first | Breast biopsy | | RNA | aaRNA^{\|\|} | Ratio |
| | Case ID | Diagnosis | birth | diagnosis | Parity status | [ng]^{§} | [ng] | 260/280 |
| 1 | 81719 | 50 | 33 | Fibrocystic changes | Parous | 28.90 | 997.80 | 1.99 |
| | | | | | Control | | | |
| 2 | 84453 | 59 | 25 | Ductal hyperplasia | Parous | 58.10 | 896.10 | 2.01 |
| | | | | | Control | | | |
| 3 | 110857 | 55 | 22 | Fibroadenoma | Parous | 54.60 | 938.20 | 2.02 |
| | | | | | Control | | | |
| 4 | 119747 | 61 | 25 | Fibrocystic changes | Parous | 25.90 | 961.10 | 2.04 |
| | | | | | Control | | | |
| 5 | 131682 | 55 | 17 | Ductal hyperplasia, | Parous | 25.80 | 725.10 | 1.99 |
| | | | | mild | Control | | | |
| 6 | 134134 | 61 | 34 | Fibroadenoma, | Parous | 80.90 | 6072.00 | 2.00 |
| | | | | adenosis | Control | | | |
| 7 | 135125 | 52 | 31 | Adenosis, ductal | Parous | 224.70 | 1483.30 | 2.03 |
| | | | | ectasia | Control | | | |
| 8 | 135447 | 77 | 23 | Apocrine metaplasia | Parous | 28.90 | 865.00 | 2.05 |
| | | | | | Control | | | |
| 9 | 135990 | 64 | 27 | Adenosis | Parous | 47.90 | 901.40 | 2.03 |
| | | | | | Control | | | |
| 10 | 136383 | 71 | 24 | Adenosis | Parous | 46.50 | 3823.00 | 2.02 |
| | | | | | Control | | | |
| 11 | 136880 | 59 | 20 | Papilloma | Parous | 39.70 | 968.30 | 2.03 |
| | | | | | Control | | | |
| 12 | 137340 | 63 | 18 | Ductal hyperplasia, | Parous | 35.80 | 235.40 | 2.01 |
| | | | | mild | Control | | | |
| 13 | 139641 | 61 | 21 | Stromal fibrosis | Parous | 49.70 | 1468.20 | 1.99 |
| | | | | | Control | | | |
| 14 | 141007 | 77 | 21 | Adenosis | Parous | 40.60 | 1283.50 | 2.02 |
| | | | | | Control | | | |
| 15 | 141300 | 78 | 24 | Adenosis | Parous | 161.90 | 2358.10 | 2.02 |
| | | | | | Control | | | |
| 17 | 143793 | 72 | 27 | Adenosis | Parous | 20.30 | 439.30 | 2.01 |
| | | | | | Control | | | |
| 18 | 148115 | 60 | 20 | Benign breast disease | Parous | 46.30 | 412.40 | 2.00 |
| | | | | | Control | | | |
| 19 | 131453 | 71 | 17 | Invasive Ductal | Parous Case | 52.50 | 2115.60 | 2.03 |
| | | | | carcinoma | | | | |
| 20 | 132370 | 61 | 26 | Invasive Ductal | Parous Case | 20.10 | 65.00 | 2.05 |
| | | | | Carcinoma | | | | |
| 21 | 132452 | 55 | 26 | Invasive Ductal and | Parous Case | 51.10 | 1225.80 | 2.07 |
| | | | | lobular carcinoma | | | | |
| 22 | 132454 | 60 | 25 | Invasive ductal | Parous Case | 46.30 | 544.40 | 2.02 |
| | | | | carcinoma | | | | |
| 23 | 132456 | 72 | 19 | Invasive ductal | Parous Case | 100.50 | 53.20 | 2.03 |
| | | | | carcinoma | | | | |
| 24 | 133360 | 57 | 19 | Invasive ductal | Parous Case | 54.90 | 1534.00 | 2.01 |
| | | | | carcinoma | | | | |
| 25 | 133931 | 74 | 26 | Invasive ductal and | Parous Case | 22.20 | 1682.90 | 2.00 |
| | | | | lobular carcinoma | | | | |
| 26 | 134133 | 75 | 26 | Invasive ductal | Parous Case | 41.70 | 6421.80 | 2.00 |
| | | | | carcinoma | | | | |
| 27 | 154855 | 75 | 25 | Invasive ductal | Parous Case | 32.50 | 5432.00 | 2.00 |
| | | | | carcinoma | | | | |
| 28 | 135984 | 76 | 20 | Mucinous | Parous Case | 21.90 | 1443.80 | 2.00 |
| | | | | adenocarcinoma | | | | |
| 29 | 137805 | 78 | 23 | Mucinous | Parous Case | 401.40 | 1235.00 | 2.02 |
| | | | | adenocarcinoma | | | | |
| 30 | 138206 | 59 | 28 | Invasive Ductal | Parous Case | 45.40 | 1574.20 | 2.00 |
| | | | | carcinoma & DCIS† | | | | |
| 31 | 138993 | 76 | 26 | Invasive Ductal | Parous Case | 83.40 | 7473.40 | 204 |
| | | | | carcinoma | | | | |
| 32 | 139128 | 84 | 31 | Invasive ductal | Parous Case | 79.30 | 1310.20 | 2.00 |
| | | | | carcinoma | | | | |
| 33 | 140569 | 67 | 24 | Invasive ductal | Parous Case | 41.10 | 1093.00 | 2.00 |
| | | | | carcinoma | | | | |
| 34 | 141008 | 55 | 29 | Invasive ductal | Parous Case | 29.70 | 1405.60 | 2.00 |
| | | | | carcinoma & DCIS | | | | |
| 35 | 141299 | 75 | 27 | Invasive ductal | Parous Case | 42.90 | 762.30 | 2.05 |
| | | | | carcinoma & DCIS | | | | |
| 38 | 145563 | 65 | 23 | Invasive ductal | Parous Case | 17.90 | 764.00 | 2.00 |
| | | | | carcinoma | | | | |
| 39 | 145564 | 74 | 25 | Invasive ductal | Parous Case | 28.50 | 828.20 | 2.00 |
| | | | | carcinoma | | | | |
| 40 | 145565 | 62 | 28 | Invasive ductal | Parous Case | 26.10 | 682.60 | 2.02 |
| | | | | carcinoma & DCIS | | | | |
| 41 | 146980 | 65 | 26 | Invasive ductal | Parous Case | 17.10 | 411.60 | 2.02 |
| | | | | carcinoma | | | | |
| 42 | 147715 | 81 | 25 | Invasive ductal | Parous Case | 106.30 | 416.70 | 2.02 |
| | | | | carcinoma & DCIS | | | | |
| 43 | 149911 | 56 | 32 | Invasive ductal | Parous Case | 107.30 | 1425.76 | 2.02 |
| | | | | carcinoma & DCIS | | | | |
| 44 | 153163 | 82 | 30 | Invasive lobular | Parous Case | 377.90 | 1326.24 | 2.00 |
| | | | | carcinoma | | | | |
| 45 | 153556 | 65 | 30 | Invasive ductal | Parous Case | 309.10 | 1427.00 | 2.00 |
| | | | | carcinoma | | | | |
| 46 | 154250 | 76 | 20 | Invasive ductal | Parous Case | 310.10 | 1428.24 | 2.11 |
| | | | | carcinoma & DCIS | | | | |
| 47 | 155065 | 79 | 28 | Invasive ductal | Parous Case | 1003.40 | 1129.98 | 2.00 |
| | | | | carcinoma | | | | |
| 48 | 155844 | 75 | 26 | Invasive ductal | Parous Case | 1537.70 | 1430.24 | 2.06 |
| | | | | carcinoma | | | | |
| 49 | 155845 | 82 | 21 | Invasive ductal | Parous Case | 310.10 | 1531.00 | 2.00 |
| | | | | carcinoma | | | | |
| 50 | 156062 | 58 | 26 | Invasive ductal | Parous Case | 311.10 | 1432.24 | 2.08 |
| | | | | carcinoma | | | | |
| 51 | 156105 | 73 | 26 | Invasive lobular | Parous Case | 305.80 | 1233.24 | 2.00 |
| | | | | carcinoma & LCIS‡ | | | | |
| 52 | 157584 | 70 | 23 | Invasive lobular | Parous Case | 325.80 | 1434.00 | 2.20 |
| | | | | carcinoma | | | | |
| 53 | 157678 | 92 | 19 | Invasive lobular | Parous Case | 1784.00 | 1635.24 | 2.00 |
| | | | | Carcinoma | | | | |
| 54 | 158532 | 70 | 25 | Invasive ductal | Parous Case | 1655.80 | 1146.94 | 2.00 |
| | | | | carcinoma | | | | |
| 55 | 158972 | 60 | 31 | Invasive ductal | Parous Case | 966.90 | 1437.50 | 2.01 |
| | | | | carcinoma & DCIS | | | | |
| 56 | 158973 | 61 | 19 | Invasive ductal | Parous Case | 1011.90 | 1444.24 | 2.00 |
| | | | | carcinoma | | | | |
| 57 | 160038 | 60 | 16 | Invasive ductal | Parous Case | 429.80 | 1439.24 | 2.00 |
| | | | | carcinoma | | | | |
| 58 | 160039 | 66 | 30 | Invasive ductal | Parous Case | 1783.50 | 1440.00 | 2.01 |
| | | | | carcinoma & DCIS | | | | |
| 59 | 160827 | 63 | 28 | Invasive ductal | Parous Case | 355.70 | 1441.24 | 2.00 |
| | | | | carcinoma & DCIS | | | | |
| 60 | 15737 | 65 | N/A | Adenosis | Nulliparous | 579.00 | 1342.67 | 2.00 |
| | | | | | Control | | | |
| 61 | 45853 | 62 | N/A | Fibroadenoma | Nulliparous | 131.90 | 1443.24 | 2.04 |
| | | | | | Control | | | |
| 62 | 131161 | 58 | N/A | Papilloma | Nulliparous | 51.10 | 2005.80 | 2.00 |
| | | | | | Control | | | |
| 63 | 132404 | 51 | N/A | Fibroadenoma, | Nulliparous | 81.20 | 2006.80 | 2.00 |
| | | | | papilloma | Control | | | |
| 64 | 141009 | 53 | N/A | Stromal fibrosis | Nulliparous | 108.80 | 977.40 | 2.07 |
| | | | | | Control | | | |
| 65 | 143964 | 50 | N/A | Apocrine metaplasia, | Nulliparous | 56.60 | 618.10 | 2.00 |
| | | | | stromal fibrosis | Control | | | |
| 66 | 149204 | 58 | N/A | Adenosis | Nulliparous | 31.10 | 401.60 | 2.00 |
| | | | | | Control | | | |
| 67 | 132372 | 53 | N/A | Invasive ductal | Nulliparous | 20.90 | 557.50 | 2.05 |
| | | | | carcinoma | Case | | | |
| 68 | 132382 | 68 | N/A | Invasive ductal and | Nulliparous | 27.60 | 386.60 | 2.00 |
| | | | | lobular carcinoma | Case | | | |
| 69 | 132402 | 77 | N/A | Invasive ductal | Nulliparous | 776.70 | 387.60 | 2.00 |
| | | | | carcinoma | Case | | | |
| 70 | 136596 | 74 | N/A | Invasive ductal | Nulliparous | 51.70 | 891.60 | 1.99 |
| | | | | carcinoma | Case | | | |
| 71 | 142667 | 87 | N/A | Invasive ductal | Nulliparous | 37.10 | 1217.50 | 2.00 |
| | | | | carcinoma | Case | | | |
| 72 | 144166 | 57 | N/A | Invasive ductal | Nulliparous | 646.70 | 1218.50 | 2.00 |
| | | | | carcinoma | Case | | | |
| 73 | 155958 | 57 | N/A | Invasive lobular | Nulliparous | 150.50 | 1219.50 | 2.08 |
| | | | | carcinoma | Case | | | |
| 74 | 156622 | 55 | N/A | Invasive ductal | Nulliparous | 433.60 | 1220.50 | 2.00 |
| | | | | carcinoma | Case | | | |

*Groups of Patients: Parous Controls, women with benign breast biopsies; Parous Cases, women with breast cancer; Nulliparous Control, childless women with benign breast biopsies, and Nulliparous Cases, Childless women with breast cancer.
^{†}DCIS, Ductal carcinoma in situ
^{‡}LCIS, Lobular carcinoma in situ
^{§}RNA [ng], total amount of RNA in nanograms obtained by LCM from each sample.
^{||}aaRNA, amplified RNA

cDNA Human Microarray Analysis

RNA isolation and amplification from LCM samples were performed as previously described [27]. Microarrays were prepared by the Fox Chase Cancer Center NCI-supported Microarray Facility. Mirror glass slides were utilized for robotically spotting 40,000 cDNAs representing 28,000 distinct human transcripts, 10,000 identified by ESTs, and 2,000 controls and blank spots. Probe construction using direct labeling with random hexamer primer and purification using the QIA-quick PCR purification kit (Qiagen) were performed as previously described [27]. After the last centrifugation at 13,000 rpm for 1 min the concentration of the eluted material was determined, then partially dried in a vacuum centrifuge and resuspended in 15 μl of hybridization buffer containing 20× saline-sodium citrate (SSC) and 0.6 μl of 10% (wt/vol) SDS. Thereafter the probes were denatured at 95° C., centrifuged for 3 minutes at 13,000 rpm and the products were pipetted onto pre-hybridized arrays; the slides were coverslipped and placed in hybridization chambers (Gene Machine). Arrays were incubated in a 42° C. water bath for 16-18 h, and subsequently washed with 0.5×SSC, 0.01% (wt/vol) SDS, followed by 0.06×SSC, at room temperature for 10 min each. The slides were centrifuged for 8 min at 800 rpm (130 g) at room temperature. The glass microarrays were hybridized placing in the red channel (labeled with Cy5) the amplified RNA from the breast samples and in the green channel (labeled with Cy3) the human universal reference amplified RNA (Stratagene Technologies, Inc., La Jolla, Calif.). Each hybridization compared Cy5-labelled cDNA reverse transcribed from amplified RNA isolated from each patient with the Cy3-labelled cDNA reverse transcribed from a universal human reference amplified RNA sample. Equal amounts of fluorescent probes were used to hybridize the cDNA microarrays in triplicate and after quality verification in the Nanodrop, replicates from the same sample were combined an re-distributed into 3 separate tubes in order to have identical replicates. Arrays were read in an Affymetrix 428 fluorescent scanner (MWG, CA) at 10 μm resolution, with variable voltage of the photomultiplier tube (PMT) for obtaining the maximal signal intensities with <1% (wt/vol) probe saturation. The resulting images were analyzed using ImaGene software version 4,2 (Biodiscovery, El Segundo, Calif.).

### Data Analysis

Normalization and statistical analysis of the expression data were carried out using Linear Models for Microarray Data (LIMMA) [28-30]. For detecting the differential expression of genes that might not necessarily be highly expressed, background correction using the “normexp” method in LIMMA was performed for adjusting the local median background estimates, a correction strategy that avoids problems with background estimates that are greater than foreground values and ensures that there were no missing or negative corrected intensities. An offset of 100 was used for both channels to further damp down the variability of log-ratios for low-intensity spots. The resulting log-ratios were normalized by using the print-tip group Lowess method with span 0.4, as recommended by Smyth et al. [30].

Moderated t-statistic was used as the basic statistic for significance analysis; it was computed for each probe and for each contrast [30]. False discovery rate (FDR) was controlled using the BH adjustment of Benjamini and Hochberg [31,32]. All genes with p value below a threshold of 0.05 were selected as differentially expressed (DE), maintaining the proportion of false discoveries in the selected group below the threshold value, in this case 5% [33]. Hierarchical clustering was done using GeneSight software (BioDiscovery Inc., El Segundo, Calif.) version 2.4).

### Gene Validation by RT-PCR Amplification

Genes that were found to be upregulated in the parous control breast were validated by real time RT-PCR using nucleotide sequences that were found using the gene accession number obtained from the cDNA glass microarrays and searching the NCI-Blast website on the world wide web at ncbi.nlm.nih.gov/BLAST/. Taqman primer and probe sets sequences are listed in Table 3. The sense and antisense primer sequences were designed using Primer3 software found on the world wide web at //frodo.wi.mit.edu/cgi-bin/primer3/primer3_www.cgi, and synthesized by the DNA Sequencing Facility at the Fox Chase Cancer Center. A beta actin primer was included as a control for gene expression. Primers were labeled with SyBroGreen dye (Applied Biosystems, Foster City, Calif.); for avoiding competition in the multiplex PCR reaction tube primer concentrations were limited and standardized. All RT-PCR reactions were performed on the ABI Prism 7000 Sequence Detection System using the fluorescent SyBro Green methodology (SYBRO Green Rt-PCR Master Mix Reagents, all from Applied Biosystems (Foster City, Calif.). For each RT-PCR reaction 100 ng of amplified RNA in a total volume of 50 μl were used. Primer and probe concentrations for target genes were optimized according to the manufacturer's recommended procedure. The following thermal cycling conditions were used: 30 min at 48° C., 10 min at 95° C., and 40 cycles of 15 seconds, denaturalization at 95° C. for 60 seconds and annealing at 60° C. Each gene was analyzed in triplicate, normalized against beta actin and expressed in relation to a calibrator sample. Results were expressed as relative gene expression (RGE) using the Ct method, as previously described [27].

### Results

### Identification of Differentially Expressed Genes in Breast Epithelium

For the analysis of the effect of parity on the genomic profile of epithelial cells from Lob 1, cDNA microarray expression profiling of the 74 breast tissue samples described in Table 1 was performed. Genes whose expression changes differed by at least 1.2-fold and that were considered to be statistically significant between nulliparous and parous women with and without cancer using established algorithms were selected for further analysis [32]. A total of 2,541 gene sequences were found to be differentially expressed (t test with false discovery rate p<0.05) in the breast epithelium of the parous control group in comparison with nulliparous control and cases and parous cases. The parous control group had 126 genes up-regulated and 103 downregulated (Table 2) with respect to the nulliparous control and case groups and to the parous group with breast cancer (cases).

TABLE 2
Genes Differentially Expressed in the Breast Epithelium of Parous Control Women

| | | | | Molecular | | Fold |
|---|---|---|---|---|---|---|
| | | | | Function | | increase/ |
| Gene name | Gene ID | Symbol | GO number | GO number | Adj P | decrease |
| Apoptosis | | | | | | |
| BCL2-associated X protein | AI565203 | BAX | GO: 0006915 | GO: 0005515 | 0.0230 | 2.6500 |
| TIA1 cytotoxic granule-associated RNA | R82978 | TIA1 | GO: 0006915 | GO: 0000166 | 0.0173 | 1.5600 |
| binding protein | | | | | | |
| TNF receptor-associated factor 1 | R71691 | TRAF1 | GO: 0006915 | GO: 0006461 | 0.017 | 1.7200 |
| TNFRSF1A-associated via death domain | AA916906 | TRADD | GO: 0006915 | GO: 0005515 | 0.027 | 1.4200 |
| CASP2 and RIPK1 | AA285065 | CRADD | GO: 0042981 | GO: 0005515 | 0.0041 | 1.8900 |
| Protein phosphatase 1F | AA806330 | PPM1F | GO: 0006915 | GO: 0016787 | 0.0142 | 1.3500 |
| Programmed cell death | AA416757 | PDCD5 | GO: 0006915 | GO: 0005554 | 0.0013 | −2.1500 |
| Mdm4 | AI310969 | MDM4 | GO: 0006915 | GO: 0004842 | 0.0134 | −1.2500 |
| Antiapoptosis | | | | | | |
| Baculoviral IAP repeat-containing 6 | H10434 | BIRC6 | GO: 0006916 | GO: 0004840 | 0.0136 | −1.2600 |
| BCL2-associated athanogene 4 | H22928 | BAG4 | GO: 0006916 | GO: 0005057 | 0.0267 | −1.2700 |
| Cell adhesion | | | | | | |
| Sema domain | AA436152 | SEMA5A | GO: 0007155 | GO: 0004872 | 0.050 | 1.810 |
| Intercellular adhesion molecule 3 | W95068 | ICAM3 | GO: 0016337 | GO: 0005178 | 0.0199 | 1.7000 |
| Epithelial V-like antigen 1 | AA668897 | EVA1 | GO: 0007155 | GO: 0005515 | 0.0110 | 1.2500 |
| Fibulin 5 | H17615 | FBLN5 | GO: 0007160 | GO: 0004888 | 0.0100 | 1.7900 |
| Formin binding protein 4 | N49573 | FNBP4 | GO: 0007155 | GO: 0005198 | 0.0279 | 1.2900 |
| Sidekick homolog 1 (chicken) | N23940 | SDK1 | GO: 0007155 | GO: 0005515 | 0.0267 | 1.2600 |
| Neuropilin 1 | AA098867 | NRP1 | GO: 0007155 | GO: 0004872 | 0.0170 | 1.2500 |
| Down syndrome cell adhesion molecule | N53145 | DSCAM | GO: 0007155 | GO: 0005515 | 0.0126 | −2.1000 |
| Discs, large homolog 5 (Drosophila) | AA478949 | DLG5 | GO: 0016337 | GO: 0005515 | 0.0136 | −1.8000 |
| Collagen, type XVI, alpha 1 | AA088202 | COL16A1 | GO: 0007155 | GO: 0005198 | 0.0102 | −1.7800 |
| Laminin, gamma 1 (formerly LAMB2) | AA599005 | LAMC1 | GO: 0007155 | GO: 0005515 | 0.0015 | −2.9100 |
| Cell signaling-signal transduction | | | | | | |
| Egf-like module containing | AI174266 | EMP2 | GO: 0007165 | GO: 0004872 | 0.0115 | 1.5100 |
| Ankyrin 2, neuronal | AI018106 | ANK2 | GO: 0007165 | GO: 0005200 | 0.0142 | 1.2500 |
| Insulin receptor substrate 1 | AA456306 | IRS1 | GO: 0007165 | GO: 0004871 | 0.0220 | 1.2900 |
| Cornichon homolog 2 (Drosophila) | R42919 | CNIH2 | GO: 0007242 | GO: 0005554 | 0.0108 | 1.2500 |
| Chimerin (chimaerin) 2 | AI026829 | CHN2 | GO: 0007242 | GO: 0005096 | 0.0111 | 1.3000 |
| Low density lipoprotein receptor-related protein 5 | R83038 | LRP5 | GO: 0016055 | GO: 0004872 | 0.014 | 1.4000 |
| G protein-coupled receptor kinase interactor 1 | AI079118 | GIT1 | GO: 0008277 | GO: 0005096 | 0.0128 | 1.3500 |
| Galanin receptor 2 | N75473 | GALR2 | GO: 0007186 | GO: 0004966 | 0.0302 | 1.2000 |
| Neuropeptide Y receptor Y1 | R43817 | NPY1R | GO: 0007165 | GO: 0001584 | 0.019 | −1.460 |
| Rap guanine nucleotide exchange factor (GEF) 6 | AA911005 | RAPGEF6 | GO: 0007264 | GO: 0005085 | 0.0020 | −1.4000 |
| RAB27A, member RAS oncogene family | AI309109 | RAB27A | GO: 0007264 | GO: 0000166 | 0.0054 | −1.8454 |
| Coiled-coil domain containing 132 | R49442 | CCDC132 | GO: 0000160 | GO: 0000155 | 0.0020 | −3.8868 |
| BRCA2 and CDKN1A interacting protein | AI033172 | BCCIP | GO: 0000079 | GO: 0005554 | 0.0070 | −1.3792 |
| Small inducible cytokine subfamily E | H05323 | SCYE1 | GO: 0007267 | GO: 0005125 | 0.0020 | −3.8681 |
| Neuropeptide S receptor 1 | H91700 | NPSR1 | GO: 0007165 | GO: 0004872 | 0.0140 | −1.4600 |
| Ankyrin repeat and death domain containing 1A | AI053438 | ANKDD1A | GO: 0007165 | GO: 0005515\| | 0.0097 | −2.3100 |
| Endothelin receptor type A | AA909960 | EDNRA | GO: 0007186 | GO: 0001599 | 0.0119 | −1.4500 |
| GIPC PDZ domain containing family, member 1 | AI094796 | GIPC1 | GO: 0007186 | GO: 0005515 | 0.0112 | −1.5500 |
| Development and differentiation | AI054096 | DDEF2 | GO: 0043087 | GO: 0005096 | 0.0130 | −1.3000 |
| enhancing factor 2 | | | | | | |
| Cell cycle and growth | | | | | | |
| DnaJ (Hsp40) homolog, subfamily A, member 2 | AI273537 | DNAJA2 | GO: 0000074 | GO: 0008270 | 0.0020 | 1.5600 |
| Homeodomain interacting protein kinase 2 | N38891 | HIPK2 | GO: 0000074 | GO: 0000166 | 0.0079 | 1.8700 |
| Retinoblastoma binding protein 6 | AA398302 | RBBP6 | GO: 0000074 | GO: 0003676 | 0.0120 | 1.5800 |
| Dynactin 1 (p150, glued homolog, Drosophila) | AA488168 | DCTN1 | GO: 0007067 | GO: 0003774 | 0.011 | 1.4800 |
| Katanin p60 subunit A 1 | T47614 | KATNA1 | GO: 0007049 | GO: 0000166 | 0.0056 | −2.6600 |
| Transforming, acidic coiled-coil containing | AA598796 | TACC1 | GO: 0007049 | GO: 0005515 | 0.0079 | −2.1700 |
| protein 1 | | | | | | |
| Sestrin 3 | AI190194 | SESN3 | GO: 0007050 | GO: 0005554 | 0.0028 | −1.9000 |
| G1 to S phase transition 1 | R62452 | GSPT1 | GO: 0000082 | GO: 0000166 | 0.0000 | −3.5000 |
| Protein phosphatase 2 | H09640 | PPP2R1B | GO: 0000074 | GO: 0000158 | 0.0199 | −2.3500 |
| LATS, large tumor suppressor, homolog 1 | AI023733 | LATS1 | GO: 0000086 | GO: 0000166 | 0.0092 | −1.9800 |
| Transmembrane and coiled-coil domains 7 | AI057241 | TMCO7 | GO: 0007076 | GO: 0005488 | 0.0110 | −1.6000 |
| Response to exogenous agents | | | | | | |
| Retinol dehydrogenase 11 (all-trans/9-cis/11-cis) | H82421 | RDH11 | GO: 0008152 | GO: 0016491 | 0.0168 | 1.6428 |
| Epoxide hydrolase 1, microsomal (xenobiotic) | AA838691 | EPHX1 | GO: 0006805 | GO: 0004301 | 0.012 | 1.7800 |
| Thioredoxin reductase 1 | AA464849 | TXNRD1 | GO: 0045454 | GO: 0015036 | 0.006 | 1.9200 |
| Immunoglobulin (CD79A) binding protein 1 | AA463498 | IGBP1 | GO: 0042113 | GO: 0008601 | 0.005 | 1.3800 |
| Calcium binding atopy-related autoantigen 1 | AA992324 | CBARA1 | GO: 0006952 | GO: 0005509 | 0.0135 | 1.3800 |
| Toll-interleukin 1 receptor | AI279454 | TIRAP | GO: 0006954 | GO: 0004888 | 0.0097 | 1.3800 |
| Scavenger receptor class A, member 3 | R10675 | SCARA3 | GO: 0006979 | GO: 0005044 | 0.0250 | 1.3900 |
| Glutathione S-transferase theta 1 | T64869 | GSTT1 | GO: 0006950 | GO: 0004364 | 0.0139 | 1.2400 |
| Epoxide hydrolase 1, microsomal (xenobiotic) | AA838691 | EPHX1 | GO: 0006805 | GO: 0004301 | 0.012 | 1.2500 |
| Chromosome 10 open reading frame 59 | AI093491 | C10orf59 | GO: 0006725 | GO: 0004497 | 0.0130 | 1.9300 |
| N-acetyltransferase 2 (arylamine N- | AI460128 | NAT2 | GO: 0006152 | GO: 0004060 | 0.0095 | 1.5000 |
| acetyltransferase) | | | | | | |
| Cell transport | | | | | | |
| Translocated promoter region | AA064778 | TPR | GO: 0006810 | GO: 0005554 | 0.0069 | 1.5300 |
| Translocation protein 1 | T98628 | TLOC1 | GO: 0015031 | GO: 0004872 | 0.0120 | 1.6300 |
| UDP-N-acetyl-alpha-D-galactosamine | AA598949 | GALNT10 | GO: 0005794 | GO: 0003779 | 0.0071 | 1.4700 |
| Armadillo repeat containing 1 | AA490502 | ARMC1 | GO: 0030001 | GO: 0046672 | 0.0122 | 1.6500 |
| Transient receptor potential cation channel | AI167481 | TRPM1 | GO: 0006812 | GO: 0005262 | 0.0144 | 1.2400 |
| Chloride channel 6 | H72322 | CLCN6 | GO: 0006811 | GO: 0005247 | 0.0220 | 1.3500 |
| HIV-1 Rev binding protein | AA927604 | HRB | GO: 0006406 | GO: 0003677 | 0.0124 | 1.4000 |
| SH3KBP1 binding protein 1 | AA457723 | SHKBP1 | GO: 0006813 | GO: 0005216 | 0.0232 | 1.6300 |
| Tweety homolog 1 (Drosophila) | R56769 | TTYH1 | GO: 0006826 | GO: 0005381 | 0.0232 | 1.6000 |
| Solute carrier family 19, member 3 | AA707858 | SLC19A3 | GO: 0006810 | GO: 0005386 | 0.0232 | 1.6400 |
| Solute carrier family 22 | AA705565 | SLC22A9 | GO: 0006810 | GO: 0005215 | 0.0119 | 1.5600 |
| Stonin 2 | AA992626 | STON2 | GO: 0006896 | GO: 0005515 | 0.0056 | −1.7224 |
| Gamma-aminobutyric acid (GABA) A receptor | R43452 | GABRB3 | GO: 0006811 | GO: 0004890 | 0.0042 | −2.3657 |
| Ficotin (collagen/fibrinogen domain containing) 1 | AI349250 | FCN1 | GO: 0006817 | GO: 0003823 | 0.0084 | −1.6600 |
| Ras-GTPase-activating protein SH3-domain- | AA598628 | G3BP | GO: 0006810 | GO: 0000166 | 0.0020 | −3.8192 |
| binding | | | | | | |
| Frequenin homolog (Drosophila) | H16821 | FREQ | GO: 0005794 | GO: 0005509 | 0.0174 | −1.4241 |
| Acyl-Coenzyme A oxidase 1, palmitoyl | AI079148 | ACOX1 | GO: 0006118 | GO: 0003995 | 0.0101 | −1.5300 |
| Cytochrome b5 reductase 4 | AI053851 | CYB5R4 | GO: 0006118 | GO: 0004128 | 0.0139 | −1.7000 |
| RAP1B, member of RAS oncogene family | AA598864 | RAP1B | GO: 0006886 | GO: 0005525 | 0.0142 | −1.8100 |
| Dysbindin domain containing 2 | AA598970 | DBNDD2 | GO: 0015031 | GO: 0005515 | 0.0247 | −1.3100 |
| Solute carrier family 20 (phosphate transporter) | AA933776 | SLC20A2 | GO: 0006810 | GO: 0004872 | 0.0108 | −1.7100 |
| Kelch-like 2, Mayven (Drosophila) | AI348818 | KLHL2 | GO: 0006888 | GO: 0005515 | 0.0108 | −2.0500 |
| Sorting nexin 11 | H16467 | SNX11 | GO: 0007242 | GO: 0005554 | 0.0247 | −1.4600 |
| Chromatin Modification | | | | | | |
| Histone cluster 1, H2ac | N50797 | HIST1H2AC | GO: 0007001 | GO: 0003677 | 0.027 | 1.2700 |
| SET domain containing 1A | AA459896 | SETD1A | GO: 0016568 | GO: 0003723 | 0.0161 | 1.8700 |
| Development-Morphogenesis | | | | | | |
| Dishevelled, dsh homolog 2 (Drosophila) | R38325 | DVL2 | GO: 0007275 | GO: 0004871 | 0.0077 | 2.066903$$ |
| Latent transforming growth factor beta binding | R87406 | LTBP4 | GO: 0007275 | GO: 0008083 | 0.0077 | 1.990103$$ |
| protein 4 | | | | | | |
| Dopey family member 2 | W15495 | DOPEY2 | GO: 0007275 | GO: 0005554 | 0.0120 | 2.5697 |
| Ephrin-B3 | AA485665 | EFNB3 | GO: 0030154 | GO: 0005005 | 0.0002 | 1.6402 |
| DiGeorge syndrome critical region gene 14 | AI369125 | DGCR14 | GO: 0007399 | GO: 0005554 | 0.0043 | 2.3211 |
| Fibroblast growth factor 11 | AA936128 | FGF11 | GO: 0007399\| | GO: 0008083 | 0.0045 | 2.2619 |
| Twist homolog 1 | AI220198 | TWIST1 | GO: 0009653 | GO: 0003677 | 0.0037 | −1.2500 |
| Protein inhibitor of activated STAT, 2 | AI151206 | PIAS2 | GO: 0007275 | GO: 0003677 | 0.0013 | −1.5000 |
| Microtubule-associated protein 1B | H17493 | MAP1B | GO: 0007517 | GO: 0005198 | 0.0036 | −2.6405 |
| Dual specificity phosphatase 22 | AA454636 | DUSP22 | GO: 0000188 | GO: 0008138 | 0.0067 | −1.6400 |
| Tropomyosin 3 | AA206591 | TPM3 | GO: 0007517 | GO: 0003779 | 0.0027 | −2.2300 |
| Cysteine rich transmembrane BMP regulator 1 | R78638 | CRIM1 | GO: 0007399 | GO: 0004867 | 0.0126 | −1.3500 |
| Split hand/foot malformation (ectrodactyly) type 1 | R38516 | SHFM1 | GO: 0030326 | GO: 0005515 | 0.0121 | −1.7200 |
| Hepatic leukemia factor | R59192 | HLF | GO: 0007275\| | GO: 0003690 | 0.0126 | −1.5000 |
| Bruno-like 4, RNA binding protein (Drosophila) | R52541 | BRUNOL4 | GO: 0009790 | GO: 0003676 | 0.0143 | −1.3400 |
| DNA repair and replication | | | | | | |
| RAD51-like 3 (S. cerevisiae) | N29765 | RAD51L3 | GO: 0006284 | GO: 0005524 | 0.024 | 1.9200 |
| Excision repair cross-complementing rodent | N49276 | ERCC6 | GO: 0006281 | GO: 0003702 | 0.0407 | 1.2500 |
| repair deficiency | | | | | | |
| Polymerase (DNA-directed) | AI017254 | POLD3 | GO: 0000731 | GO: 0003891 | 0.0073 | 1.5000 |
| Ankyrin repeat domain 17 | R37816 | ANKRD17 | GO: 0006298 | GO: 0003676 | 0.555 | 1.780 |
| Translin | AA460927 | TSN | GO: 0006310 | GO: 0003677 | 0.065 | 1.940 |
| Nth endonuclease III-like 1 (E. coli) | AI369190 | NTHL1 | GO: 0006284 | GO: 0003677 | 0.0094 | 1.9200 |
| Three prime repair exonuclease 1 | AI352447 | TREX1 | GO: 0006281 | GO: 0003697 | 0.0114 | 1.5400 |
| Ubiquitin-activating enzyme E1 | AA598670 | UBE1 | GO: 0006260 | GO: 0000166 | 0.044 | −1.250 |
| Structural maintenance of chromosomes 2 | AA598549 | SMC2 | GO: 0000067 | GO: 0000166 | 0.0163 | −1.6100 |
| Miscellaneous processes | | | | | | |
| Carcinoembryonic antigen-related cell adhesion | AI242105 | CEACAM4 | GO: 0005887 | GO: 0005554 | 0.01 | 1.6500 |
| Diaphanous homolog 3 (Drosophila) | AI018026 | DIAPH3 | GO: 0016043 | GO: 0003779 | 0.0045 | 2.2219 |
| Sarcospan (Kras oncogene-associated gene) | AA458998 | SSPN | GO: 0006936 | GO: 0005554 | 0.0065 | 1.5900 |
| Thrombospondin, type I, domain containing 4 | AA120866 | THSD4 | GO: 0031012 | GO: 0008233 | 0.0097 | 1.8500 |
| Annexin A5 | AI269079 | ANXA5 | GO: 0007596 | GO: 0004859 | 0.0070 | −1.3938 |
| Lactamase, beta | AI273225 | LACTB | GO: 0046677 | GO: 0016787 | 0.0027 | −1.8100 |
| RNA processing | | | | | | |
| DEAD (Asp-Glu-Ala-Asp) box polypeptide 17 | H82870 | DDX17 | GO: 0006396 | GO: 0008026 | 0.003 | 3.024 |
| Eukaryotic translation initiation factor 4A, isoform 3 | N79030 | EIF4A3 | GO: 0006364 | GO: 0005524 | 0.021 | 1.710 |
| Tetratricopeptide repeat domain 8 | W37689 | TTC8 | GO: 0007600 | GO: 0005488 | 0.0162 | 1.8722 |
| Pseudouridylate synthase 7 homolog (S. cerevisiae) | AA434411 | PUS7 | GO: 0008033 | GO: 0004730 | 0.0176 | 1.4400 |
| Processing of precursor 7 | H71218 | POP7 | GO: 0008033 | GO: 0003676 | 0.010 | 1.540 |
| Survival of motor neuron protein interacting | N26026 | SIP1 | GO: 0000245 | GO: 0031202 | 0.0173 | 1.7400 |
| protein 1 | | | | | | |
| Splicing factor, arginine/serine-rich 10 | AI583623 | SFRS10 | GO: 0000398 | GO: 0000166 | 0.0027 | −2.2000 |
| BMS1-like, ribosome assembly protein (yeast) | AA915891 | BMS1L | GO: 0007046 | GO: 0000166 | 0.0022 | −1.6900 |
| Brix domain containing 2 | AI025116 | BXDC2 | GO: 0007046 | GO: 0005554 | 0.0005 | −2.1000 |
| Metabolism | | | | | | |
| Heparan sulfate (glucosamine) 3-O- | AA973808 | HS3ST4 | GO: 0030201 | GO: 0008467 | 0.046 | 1.980 |
| sulfotransferase 4 | | | | | | |
| Dihydrolipoamide branched chain transacylase E2 | AI004719 | DBT | GO: 0008152 | GO: 0005515 | 0.0029 | 2.9621 |
| Homer homolog 1 (Drosophila) | AA903860 | HOMER1 | GO: 0007206 | GO: 0005515 | 0.0051 | 2.0195 |
| SID1 transmembrane family, member 2 | T98941 | SIDT2 | GO: 0016042 | GO: 0003847 | 0.0180 | 1.2718 |
| Fumarylacetoacetate hydrolase | AA010559 | FAH | GO: 0006559 | GO: 0000287 | 0.0282 | 1.2900 |
| Dehydrodolichyl diphosphate synthase | AA995913 | DHDDS | GO: 0008152 | GO: 0016740 | 0.0092 | 1.9600 |
| Acyl-CoA synthetase short-chain family member 1 | N67766 | ACSS1 | GO: 0008152 | GO: 0003824 | 0.0220 | 1.7600 |
| Fumarylacetoacetate hydrolase | AA010559 | FAH | GO: 0006559 | GO: 0004334 | 0.0282 | 1.2500 |
| (fumarylacetoacetase) | | | | | | |
| Protein tyrosine phosphatase, receptor type, B | N66422 | PTPRB | GO: 0006796 | GO: 0005529 | 0.0144 | 1.2600 |
| Amylase, alpha 1A; salivary | R64129 | AMY1A | GO: 0005975 | GO: 0004556 | 0.0094 | −1.2500 |
| Acyl-CoA synthetase long-chain family member 3 | AI206454 | ACSL3 | GO: 000663 | GO: 0003824 | 0.0124 | −1.2500 |
| Protein biosynthesis and metabolism | | | | | | |
| Protein tyrosine phosphatase, receptor type, C | AA703526 | PTPRC | GO: 0006470 | GO: 0004725 | 0.0073 | 1.3209 |
| Vacuolar protein sorting 13 homolog C (S. cerevisiae) | AA663968 | VPS13C | GO: 0006104 | GO: 0005554 | 0.0041 | 2.3952 |
| Lysyl oxidase | H80737 | LOX | GO: 0006464 | GO: 0004720 | 0.0022 | 3.6726 |
| Tubulin tyrosine ligase-like family, member 5 | R34225 | TTLL5 | GO: 0006464 | GO: 0004835 | 0.0111 | 3.2004 |
| Protein tyrosine phosphatase, non-receptor | W72293 | PTPN21 | GO: 0006470 | GO: 0004725 | 0.0282 | 1.2500 |
| type 21 | | | | | | |
| Hypothetical protein MGC42105 | AA416627 | MGC42105 | GO: 0006468 | GO: 0046872 | 0.0224 | 1.2400 |
| Ribosomal protein L9 | AI199007 | RPL9 | GO: 0006412 | GO: 0003723 | 0.0119 | 1.2600 |
| GrpE-like 1, mitochondrial (E. coli) | AA449720 | GRPEL1 | GO: 0006457 | GO: 0000774 | 0.0118 | 1.2500 |
| Beta-site APP-cleaving enzyme 2 | AA457119 | BACE2 | GO: 0006464 | GO: 0004194 | 0.0282 | 1.2400 |
| Serine/threonine/tyrosine interacting-like 1 | AI205036 | STYXL1 | GO: 0006470 | GO: 0008138 | 0.0066 | −2.6900 |
| Mitochondrial ribosomal protein S16 | AA887401 | MRPS16 | GO: 0006412 | GO: 0003735 | 0.0013 | −1.6700 |
| Eukaryotic translation initiation factor 2B | AI174400 | EIF2B1 | GO: 0006412 | GO: 0003743 | 0.0017 | −1.4500 |
| Tryptophanyl tRNA synthetase 2 (mitochondrial) | R43272 | WARS2 | GO: 0006412 | GO: 0000166 | 0.0176 | −1.7200 |
| Lipase, hepatic | AI054269 | LIPC | GO: 0006487 | GO: 0004806 | 0.0028 | −2.1300 |
| Par-3 partitioning defective 3 homolog (C. elegans) | AA902790 | PARD3 | GO: 0006461 | GO: 0005515 | 0.0028 | −1.5500 |
| Transient receptor potential cation channel | AA598596 | TRPC4AP | GO: 0006461 | GO: 0005524 | 0.0074 | −1.5700 |
| Capping protein (actin filament) muscle Z-line | W92769 | CAPZA1 | GO: 0006461 | GO: 0003779 | 0.0045 | −1.7800 |
| Collagen, type IV | AA971606 | COL4A3BP | GO: 0006468 | GO: 0004674 | 0.0139 | −1.6700 |
| Mitochondrial ribosomal protein S11 | AI032875 | MRPS11 | GO: 0006412 | GO: 0003735 | 0.0139 | −1.6700 |
| Eukaryotic translation initiation factor 1A | AI214283 | EIF1AY | GO: 0006412 | GO: 0003723 | 0.0138 | −2.1500 |
| Farnesyltransferase, CAAX box, alpha | AA283874 | FNTA | GO: 0018347 | GO: 0004660 | 0.0247 | −1.7200 |
| GrpE-like 2, mitochondrial (E. coli) | AA598831 | GRPEL2 | GO: 0006457 | GO: 0000774 | 0.0199 | −1.6100 |
| Proteolysis and ubiquitination | | | | | | |
| Cathepsin B | AI091648 | CTSB | GO: 0006508 | GO: 0004213 | 0.0046 | 2.1571 |
| Dipeptidyl-peptidase 3 | AA430361 | DPP3 | GO: 0006508 | GO: 0004177 | 0.0079 | 1.5400 |
| Peptidase D | AA481543 | PEPD | GO: 0006508 | GO: 0004251 | 0.031 | 1.420 |
| E3 ubiquitin protein ligase | W86992 | EDD1 | GO: 0006511 | GO: 0004840 | 0.0168 | 1.6632 |
| Ring finger protein 44 | AI675516 | RNF44 | GO: 0016567 | GO: 0004842 | 0.0148 | 1.3200 |
| Arginyltransferase 1 | AI015417 | ATE1 | GO: 0006512 | GO: 0004057 | 0.0097 | 1.2600 |
| Heterogeneous nuclear ribonucleoprotein R | AA779191 | HNRPR | GO: 0006397 | GO: 0000166 | 0.0137 | 1.2500 |
| Gem (nuclear organelle) associated protein 4 | AA041254 | GEMIN4 | GO: 0000398 | GO: 0005515 | 0.0282 | 1.3000 |
| SUMO-1 activating enzyme subunit 1 | AA598488 | SAE1 | GO: 0016567 | GO: 0004839 | 0.0171 | −1.5842 |
| AFG3 ATPase family gene 3-like 2 (yeast) | AI219905 | AFG3L2 | GO: 0006508 | GO: 0000166 | 0.0010 | −2.1800 |
| TRIAD3 protein | AI051657 | TRIAD3 | GO: 0006512 | GO: 0008270 | 0.0050 | −2.0472 |
| Ubiquitin specific peptidase 30 | AI055850 | USP30 | GO: 0006511 | GO: 0004197 | 0.0008 | −1.7700 |
| Ariadne homolog | AI185068 | ARIH1 | GO: 0006511 | GO: 0004842 | 0.0033 | −2.1400 |
| Ubiquitin-conjugating enzyme E2E 1 | AA197307 | UBE2E1 | GO: 0006511 | GO: 0004840 | 0.0131 | −2.0700 |
| Transcription | | | | | | |
| Bromodomain PHD finger transcription factor | AA704421 | BPTF | GO: 0000122 | GO: 0005515 | 0.066 | 2.000 |
| Suppressor of Ty 5 homolog (S. cerevisiae) | R21511 | SUPT5H | GO: 0000122 | GO: 0003711 | 0.043 | 2.150 |
| SRY (sex determining region Y)-box 10 | AA976578 | SOX10 | GO: 0006350 | GO: 0003677 | 0.0281 | 1.9300 |
| p300/CBP-associated factor | N74637 | PCAF | GO: 0006350 | 0.001368735 | 0.0500 | 1.2500 |
| Forkhead box K2 | AA136472 | FOXK2 | GO: 0006350 | GO: 0003700 | 0.0142 | 1.2550 |
| Kv channel interacting protein 3, calsenilin | H39123 | KCNIP3 | GO: 0006350 | GO: 0003677 | 0.0247 | 1.3000 |
| Protein inhibitor of activated STAT, 1 | N91175 | PIAS1 | GO: 0006350 | GO: 0003677 | 0.0280 | 1.3100 |
| Regulatory factor X-associated protein | AI365571 | RFXAP | GO: 0006366 | GO: 0003700 | 0.0139 | 1.2400 |
| Zinc finger protein 16 | H17016 | ZNF16 | GO: 0006350 | GO: 0003677 | 0.012 | 1.2340 |
| Inhibitor of DNA binding 4 | AA464856 | ID4 | GO: 0006357 | GO: 0003714 | 0.978 | 2.100 |
| General transcription factor IIB | H23976 | GTF2B | GO: 0006355 | GO: 0016251 | 0.009 | 1.53859 |
| Zinc finger protein 26 | R97944 | ZNF28 | GO: 0006355 | GO: 0003676 | 0.017 | 1.53388 |
| Zinc finger protein 496 | W94267 | ZNF498 | GO: 0006355 | GO: 0003676 | 0.0051 | 2.0074 |
| Zinc finger protein 544 | AA885085 | ZNF544 | GO: 0006355 | GO: 0003676 | 0.0102 | 1.2500 |
| Zinc finger protein 710 | AI025842 | ZNF710 | GO: 0006355 | GO: 0003676 | 0.0054 | 1.9023 |
| Bromodomain adjacent to zinc finger domain, 2A | AA699460 | BAZ2A | GO: 0006355 | GO: 0003677 | 0.015 | 1.2450 |
| Homeobox D1 | W68537 | HOXD1 | GO: 0006355 | GO: 0003700 | 0.0321 | 1.2600 |
| HIR histone cell cycle regulation defective | AA609365 | HIRA | GO: 0006357 | GO: 0003700 | 0.0220 | 1.2600 |
| homolog A | | | | | | |
| Transducin-like enhancer of split 3 (E(sp1) | AI216623 | TLE3 | GO: 0006355 | GO: 0005554 | 0.0131 | 1.2500 |
| homolog | | | | | | |
| Zinc finger protein 268 | AI277336 | ZNF268 | GO: 0006355 | GO: 0008270 | 0.0142 | 1.5000 |
| Zinc finger protein 275 | AA406125 | ZNF275 | GO: 0006355 | GO: 0003677 | 0.032 | 1.2800 |
| Histone deacetylase 8 | AI053481 | HDAC8 | GO: 0000122 | GO: 0004407 | 0.0027 | −2.2000 |
| Zinc finger protein 425 | H20279 | ZNF425 | GO: 0006355 | GO: 0003676 | 0.0054 | −1.6200 |
| PBX/knotted 1 homeobox 2 | AI024125 | PKNOX2 | GO: 0006355 | GO: 0003700 | 0.0040 | −1.6400 |
| Methyl-CpG binding domain protein 3 | AI017865 | MBD3 | GO: 0006350 | GO: 0003677 | 0.0027 | −3.1705 |
| General transcription factor IIIC | AI184450 | GTF3C4 | GO: 0006350 | GO: 0003677 | 0.0027 | −1.8700 |
| Ring finger protein 12 | AA598809 | RNF12 | GO: 0006350 | GO: 0003714 | 0.0079 | −1.2500 |
| D4, zinc and double PHD fingers family 2 | AA496782 | DPF2 | GO: 0006350 | GO: 0003676 | 0.016 | −1.690 |
| SRY (sex determining region Y)-box 3 | AI359981 | SOX3 | GO: 0006355 | GO: 0003677 | 0.00476 | −2.1142 |
| POU domain, class 6, transcription factor 1 | AI123130 | POU6F1 | GO: 0006355 | GO: 0003700 | 0.0082 | −1.3700 |
| Myeloid/lymphoid or mixed-lineage leukemia | AI197974 | MLLT6 | GO: 0006355 | GO: 0005515 | 0.0097 | −1.4100 |
| RAR-related orphan receptor A | AI022327 | RORA | GO: 0006350 | GO: 0003700 | 0.0116 | −1.4000 |
| GATA zinc finger domain containing 2A | AA458840 | GATAD2A | GO: 0006306 | GO: 0030674 | 0.0224 | −1.9700 |
| Biological process unknown | | | | | | |
| Frequenin homolog (Drosophila) | AA918755 | FREO | GO: 0000004 | GO: 0005509 | 0.008 | 1.9765 |
| Fibulin 2 | AA452840 | FBLN2 | GO: 0000004 | GO: 0005509 | 0.008 | 1.8349 |
| DEAH (Asp-Glu-Ala-Asp/His) bax polypeptide 57 | AI125363 | DHX57 | GO: 0000004 | GO: 0003697 | 0.0120 | 2.9587 |
| Zinc finger, DHHC-type containing 9 | AI346102 | ZDHHC9 | GO: 0000004 | GO: 0000186 | 0.0282 | 1.2400 |
| WD repeat domain 44 | W80619 | WDR44 | GO: 0000004 | GO: 0008270 | 0.0162 | 1.8603 |
| Ectonucleoside triphosphate | AI247824 | ENTPD3 | GO: 0000004 | GO: 0004050 | 0.0232 | 1.2600 |
| diphosphohydrolase 3 | | | | | | |
| Phosphatase and actin regulator 1 | R99333 | PHACTR1 | GO: 0000004 | GO: 0005096 | 0.0054 | −1.9118 |
| Hypothetical protein MGC4562 | AI184226 | MGC4562 | GO: 0000004 | GO: 0003723 | 0.0064 | −1.8500 |
| Heterogeneous nuclear ribonucleoprotein M | AI220112 | HNRPM | GO: 0000004 | GO: 0003723 | 0.0010 | −1.7300 |
| RB1-inducible coiled-coil 1 | R38102 | RB1CC1 | GO: 0000004 | GO: 0016301 | 0.026 | −1.300 |
| Dedicator of cytokinesis 5 | AA932511 | DOCK5 | GO: 0000004 | GO: 0005085 | 0.0042 | −1.3700 |
| DEAD (Asp-Glu-Ala-Asp) box polypeptide 46 | AI003503 | DDX46 | GO: 0000004 | GO: 0000166 | 0.0020 | −2.1900 |
| Docking protein 5 | R39924 | DOK5 | GO: 0000004 | GO: 0005158 | 0.0267 | −1.2500 |
| Progesterone receptor membrane component 2 | AA456304 | PGRMC2 | GO: 0000004 | GO: 0003707 | 0.0111 | −1.2500 |
| B-cell CLL/lymphoma 7A | H90147 | BCL7A | GO: 0000004 | GO: 0003779 | 0.0131 | −1.3000 |
| Zinc finger protein 320 | AI025436 | ZNF320 | GO: 0000004 | GO: 0003676 | 0.0124 | −1.5000 |
| Biological process and molecular function unknown | | | | | | |
| ORM1-like 1 (S. cerevisiae) | AA437132 | ORMDL1 | GO: 0000004 | GO: 0005554 | 0.0029 | 3.0238 |
| Ankyrin repeat domain 12 | AA938440 | ANKRD12 | GO: 0000004 | GO: 0005554 | 0.0054 | 1.8890 |
| Transmembrane protein 27 | AA999850 | TMEM27 | GO: 0000004 | GO: 0005554 | 0.0056 | 1.7801 |
| DKFZp434A0131 protein | AA032084 | DKFZP434A0131 | GO: 0000004 | GO: 0005554 | 0.0282 | 1.2500 |
| Neurensin 2 | AI199579 | NRSN2 | GO: 0000004 | GO: 0005554 | 0.0023 | −4.8500 |
| Transmembrane protein 32 | AA251026 | TMEM32 | GO: 0000004 | GO: 0005554 | 0.0045 | −4.4300 |
| Zinc finger, RAN-binding domain containing 1 | AI033098 | ZRANB1 | GO: 0000004 | GO: 0005554 | 0.0050 | −1.8500 |
| Vitamin K epoxide reductase complex | AI279477 | VKORC1L1 | GO: 0000004 | GO: 0005554 | 0.0105 | −1.6600 |
| Family with sequence similarity 57, member A | H23091 | FAM57A | GO: 0000004 | GO: 0005554 | 0.0247 | −3.8600 |
| Microcephaly, primary autosomal recessive 1 | AA156424 | MCPH1 | GO: 0000004 | GO: 0005554 | 0.0109 | −3.9400 |

### Hierarchical Cluster Analysis

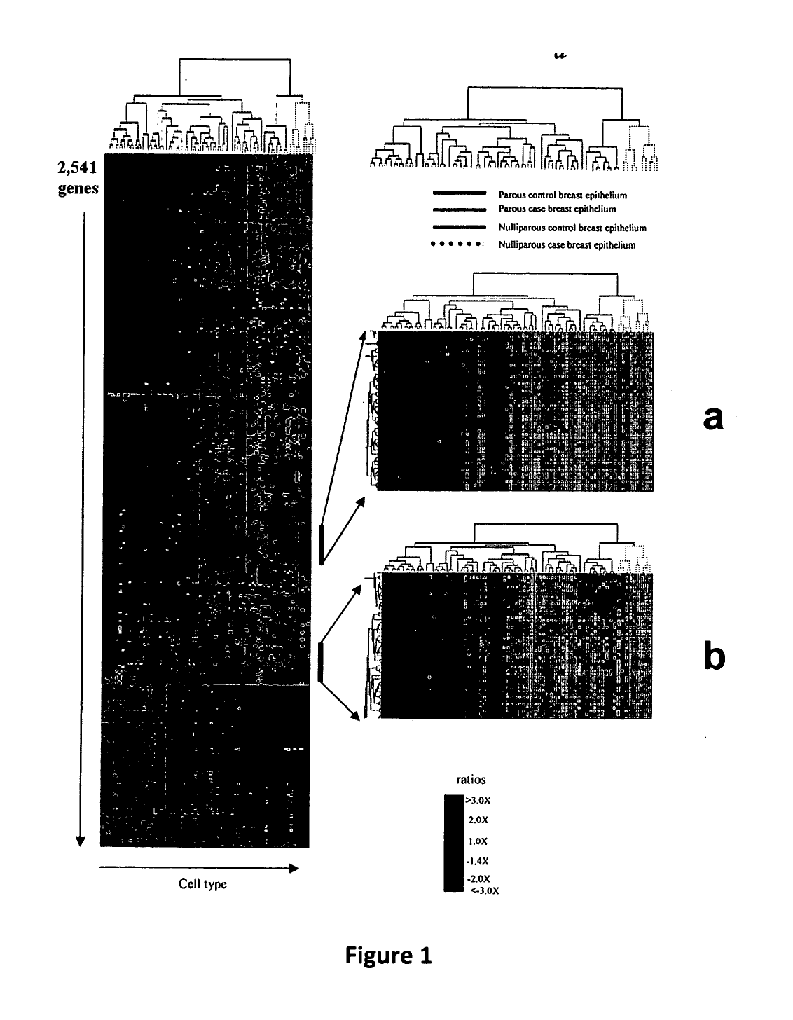

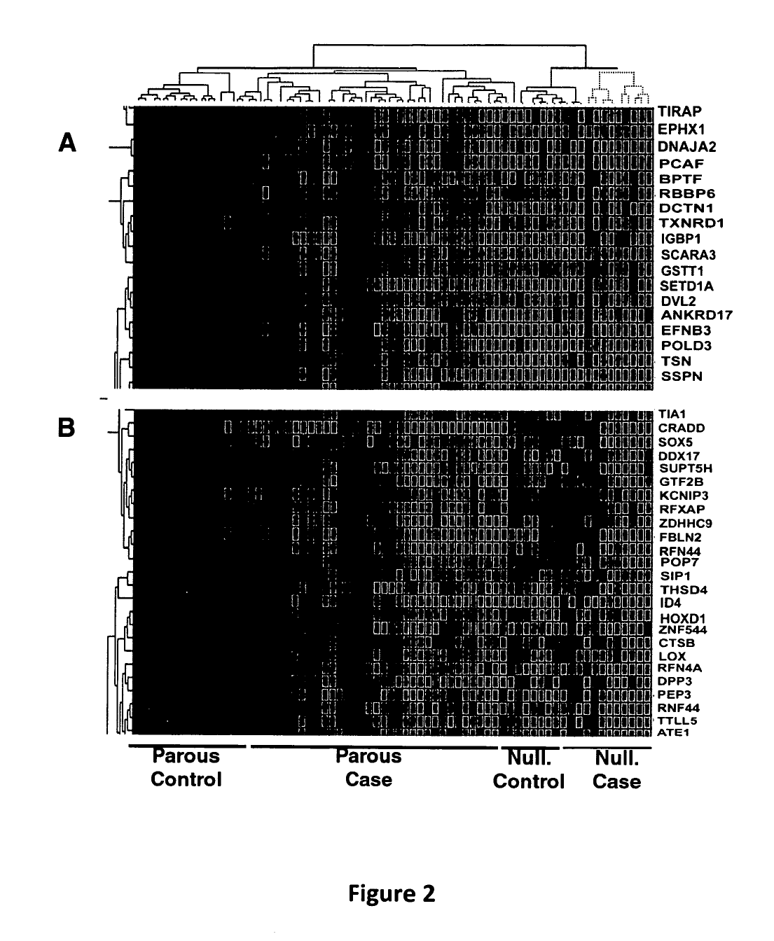

Unsupervised hierarchical clustering performed using the expression profiles of 2,541 globally varying genes across the nulliparous and parous data sets representing the four groups revealed that samples clustered primarily based on parity status (FIG. 1). This suggested that the principal source of global variation in gene expression across these data sets was due to genetic differences between women due to reproductive history. This observation suggested that determining which parity-induced gene expression changes were conserved among these highly divergent groups could represent a powerful approach to defining a parity-related gene expression signature. Results of clustering set depicted in FIGS. 2a and 2b indicate that the combined parity and absence of breast cancer data generate a distinct genomic profile that differs from the breast cancer groups, irrespective of parity history and from the nulliparous cancer free group, which has been traditionally identified as a high risk group.

### Gene Functional Category Analysis

We measured the relevance of Gene Ontology (GO) terms [34] belonging to the category of biological processes in the breast epithelium of parous women and analyzed the biological significance of those terms that were found to be deregulated in response to an early reproductive event with high statistical significance (Tables 2, 3 and 4). Among the 18 categories identified to be contain deregulated genes the most highly represented biological process was gene transcription, in which 21 genes (64%) were upregulated and 12 genes (36%) were downregulated. Higher gene expression was observed in 11 processes that included proteolysis and ubiquitination, cell adhesion, response to exogenous agents, metabolism, DNA repair and replication, RNA processing, apoptosis, miscellaneous processes, antiapoptosis, and chromatin modification, in which the ratios of up to down regulated genes ranged from 1.75 to 11 (Table 2).

A greater number of genes with lower level of expression was observed in seven processes, cell transport, protein biosynthesis and metabolism, cell signaling-signal transduction, biological process unknown, and biological process and molecular function unknown. The genes comprised in these categories are listed in Table 2. A number of genes that in the arrays of the parous control breast epithelial cells were either significantly upregulated or not modified by the reproductive process were confirmed by RT PCR. They included: TNFRSF1A-associated via death domain (TRADD), eukaryotic translation initiation factor 4A, isoform 3 (EIF4A3), Suppressor of Ty 5 homolog (S. cerevisiae) (SUPT5H) [35], SRY (sex determining region Y)-box 5 (SOX5), Carcinoembryonic antigen-related cell adhesion molecule 1 (CEACAM1), Homeobox D1 (HOXD1), Ephrin B3 (EFNB3), p300/CBPassociated factor (PCAF), Inhibitor of DNA binding 4 (ID4), and Surfeit (Table 3). All genes detected as differentially expressed by the microarray platform were confirmed to be differentially expressed by RT-PCR (p value <0.5), whereas those that did not differ among parous and nulliparous control and cases, such as Surfeit, did not differ either in the level of expression by RT-PCR (Table 3).

TABLE 3
RT-PCR validation of genes upregulated in the breast epithelium of parous women

| | | | | Nulli | | |
|---|---|---|---|---|---|---|
| | Gene | | Parous | parous | Parous | Nulli |
| Gene Name | Symbol | Primer Sequence | control | control | case | parous case |
| TNFRSF1A- | TRADD | gatggccttagggttccttc | 11488.00 ± 985.00 | 0.57 ± 0.33 | 2.18 ± 1.57 | 1.89 ± 1.85 |
| associated | | | | | | |
| via death | | | | | | |
| domain | | | | | | |
| Eukaryotic | EIF4A3 | aagaaaggtggactggctga | 3822.18 ± 764.10 | 1.09 ± 1.04 | 2.29 ± 2.72 | 12.97 ± 27.51 |
| translation | | | | | | |
| initiation | | | | | | |
| factor 4A, | | | | | | |
| isoform 3 | | | | | | |
| Suppressor | SUPT5H | ctttgaggggaaccgttaca | 1517.76 ± 234.55 | 0.26 ± 0.12 | 16.84 ± 26.09 | 2.90 ± 3.15 |
| of Ty 5 | | | | | | |
| homolog | | | | | | |
| (S. cerevisiae) | | | | | | |
| SRY (sex | SOX5 | agggactcccgagagcttag | 267.61 ± 24.87 | 0.79 ± 0.71 | 2.73 ± 3.05 | 6.04 ± 5.71 |
| determining | | | | | | |
| region Y)-box 5 | | | | | | |
| Carcinoembryonic | CEACAM1 | acccacctgcacagtactcc | 12.58 ± 1.01 | 1.97 ± 0.16 | 0.64 ± 0.06 | 1.74 ± 0.14 |
| antigen-related | | | | | | |
| cell | | | | | | |
| adhesion | | | | | | |
| molecule 1 | | | | | | |
| Homeo box D1 | HOXD1 | ttcagcaccaagcaactgac | 9.49 ± 3.15 | 2.57 ± 3.54 | 2.64 ± 2.34 | 1.11 ± 1.11 |
| Ephrin B3 | EFNB3 | cttcccaagatctcccttcc | 3.63 ± 3.23 | 1.11 ± 0.08 | 0.7 ± 0.59 | 1.17 ± 0.84 |
| p300/CBP- | PCAF | acgttcacctgctggtccaa | 98.36 ± 21.44 | 1.38 ± 0.33 | 9.87 ± 3.76 | 2.95 ± 5.34 |
| associated | | | | | | |
| factor | | | | | | |
| Inhibitor of DNA | ID4 | atgggatgaggaaatgcttg | 830.28 ± 100.33 | 0.21 ± 0.23 | 33.80 ± 63.44 | 3.37 ± 3.83 |
| binding 4 | | | | | | |
| Surfeit 5 | Surfeit | cctgcctgcaggttagaaag | 1.12 ± 1.13 | 1.75 ± 0.08 | 1.35 ± 0.67 | 1.53 ± 2.16 |

### Discussion

The present work is the first demonstration that an early first full term pregnancy imprints in the involuted breast lobules of postmenopausal parous women free of breast cancer a specific genomic signature that significantly differs from that of parous women with cancer, and nulliparous women with or without the disease. The cDNA microarray analysis of epithelial RNA of completely involuted lobules, represented by Lob 1, obtained by laser capture microdissection, revealed that these cells express a genomic signature comprised of 232 deregulated genes representing 18 functional categories.

The signature is comprised of both upregulated and downregulated genes. Deregulated genes predominated in the category of transcription, in which 63% were upregulated and 37% downregulated. The fact that the number of down-regulated genes was slightly higher in the cell transport, protein biosynthesis metabolism, cell signaling/signal transduction, development and morphogenesis, cell cycle and growth, as well as in those categories in which the biological process and the molecular functions are unknown indicates that downregulation or silencing of gene expression plays an important role in terminal differentiation [36].

Twenty three genes were found to be significantly upregulated in the parous breast epithelium in the categories of transcription and chromatin modification, an indication that modifications in transcriptional activity during pregnancy plays an important role and become a permanent component of the genomic signature imprinted by this physiological process in the postmenopausal breast epithelium greater than two-fold significant increase (p<0.05) over control values, was observed in the Bromodomain PHD finger transcription factor (BPTF), SUPT5, which has 50% similarity to yeast SPT5 and is part of a protein complex involved in transcriptional repression by modulating chromatin structure [35], and zinc finger protein 498 (ZNF498), that is involved in the regulation of nucleobase, nucleoside, nucleotide and nucleic acid metabolism. The expression of BPTF has been reported to be lost or significantly reduced in primary carcinomas and in cell lines established from different human carcinomas, supporting our postulate that this gene may play a role in suppression of tumors originating from epithelial tissue [37,38].

ID4, a member of the ID family of proteins (Id1-Id4) that function as dominant negative regulators of basic helix-loop-helix transcription factors, was increased in the parous women epithelium, as confirmed by RT-PCR that detected significant increase in the levels of expression from 0.21±0.23 in nulliparous controls to 830.28±100.33 in parous controls (Table 3). ID4 mRNA has been reported to be expressed in normal breast epithelium and myoepithelium, but to be absent in estrogen receptor alpha (ER-α) positive invasive carcinomas, in sporadic breast cancers expressing both ER-α and BRCA1 [39], in ductal carcinomas in situ(DCIS), and atypical ductal hyperplasias (ADH) [40]. Epigenetic inactivation of ID4 has been reported in human leukemia [41], colorectal cancer [42], and gastric adenocarcinoma [43]. Its complete or partial epigenetic inactivation also occurs in both ER-α positive and negative cells, i.e., T47D, MCF-7, and HBL-100, BT20, BT549, and BR2, respectively [44]. These findings support the role of this gene as a putative tumor-suppressor gene and as a key regulator of cell differentiation.

The fact that SRY (sex determining region Y)-box 10 or SOX10, a gene that is methylated in the breast cancer cell line MCF7 [45], is significantly upregulated in the breast of parous women indicates that it may play an integral role in the specification and transcription of the terminal differentiation that has been reported in other systems, such as astrocytes and oligodendrocytes [46]. In contrast, SRY (sex determining region Y)-box 3 (SOX3), that is involved in the regulation of embryonic development and determination of cell fate [47], and is essential for the maintenance of spermatogonial stem cells [48], is downregulated in the parous breast. These observations suggest that these genes might play in the breast a role similar to that described in neural and male reproductive organs, respectively.

Transcription factors also associated to co activators and chromatin remodeling, such PCAF, which we have previously found to be significantly up-regulated in breast epithelial cells of parous women [6,21,25,26], seem to play an important role in the genomic signature induced by pregnancy in breast epithelial cells. The p300/CBP family of coactivators can interact with the isolated A/B domain of the ER-α, enhancing its AF-1 activity, thus contributing to ligand-independent activity of the receptor under the stimulus of steroid receptor coactivator-1 (SRC-1) [49]. Interestingly, p300/CBP is recruited by SRC-1 and cofactors such as transcription intermediary factor 2 (TIF2) and amplified in breast cancer 1 (AIB1), that interact with nuclear receptors in a ligand-dependent manner for enhancing transcriptional activation by the receptor via histone acetylation/methylation [50]. PCAF is also a co activator of the tumor suppressor p53 and participates in p53-mediated transactivation of target genes through acetylation of both bound p53 and histones within p53 target promoters [51]. The up-regulation of PCAF in the differentiated breast epithelial cells of parous women might be associated with an increase in the protein levels of the histone acetyl transferases p300, while CBP suppresses the level of histone deacetylase and increases the level of acetylated histone H4, as it has been reported for metastatic breast cancer cells after treatment with transretinoic acid (ATRA), which also up-regulates the expression of BAX [52], a proapoptotic gene that is also up-regulated in the parous breast epithelial cells.

The general transcription factor IIB (GTF2B) that encodes one of the ubiquitous factors required for transcription initiation by RNA polymerase II and HOXD1, is also upregulated in the parous breast. Of great interest is the fact that transcription factors encoded by the HOX genes that play a crucial role in Drosophila, Xenopus, and mammalian embryonic differentiation and development, up-regulate HOXC6, HOXD1, and HOXD8 expression in human neuroblastoma cells that are chemically induced to differentiate, an indication that HOX is associated with maturation toward a differentiated neuronal phenotype [53].

Two protein inhibitors of activated STAT (PIAS) were found to be deregulated in the breast epithelium of parous women; PIAS1 was upregulated and PIAS2 (also called PIASx), was down-regulated. Members of the PIAS protein family have been identified as negative regulators of STAT signaling and of transcription factors such nuclear factor kappa B and p53 [54]. PIAS members have small ubiquitin-like modifier (SUMO) E3-ligase activity, PIAS1 exerting a direct inhibition of STAT1 DNA binding, whereas PIAS2 recruiting histone deacetylase 3 (HDAC3) for repressing STAT4-dependent transcription. Several PIAS can also cause STAT sumoylation, which is likely to inhibit STAT signaling [55]. The downregulation of PIAS2 is interesting, since the extent of PIAS and SUMO family expression in breast tissues remains unclear, although preliminary evidence suggests that dysregulation of PIAS expression does occur in human breast cancers.

Among the genes that are downregulated in the involuted lobular epithelium of postmenopausal parous women are histone deacetylase 8 (HDAC8) and methyl-CpG binding domain protein 3 (MBD3). The importance of the downregulation of these two genes is highlighted by the facts that histone deacetylases (HDACs) interact with DNA methyltransferases (DNMT) and methyl CpG-binding domain (MBD) proteins, which are associated with CpG island methylation, another epigenetic modification involved in transcriptional repression and heterochromatin remodeling [56-59]. The inhibition of HDAC by trichostatin A (TSA) induces terminal differentiation of mouse erythroleukemia cells and apoptosis of lymphoid and colorectal cancer cells. In addition, TSA treatment of cells expressing the PML zinc finger protein derepresses transcription and allows cells to differentiate normally. These findings have led to the development of HDAC inhibitors as potential agents for the treatment of certain forms of cancer [57]. Interestingly, the deacetylase activity of HDAC8 is inhibited by PKA mediated phosphorylation, resulting in the hyperacetylation of histones H3 and H4, a phenomenon similar to that induced by human chorionic gonadotropin (hCG) in the human breast [58], and that represents a novel mechanism of regulation of the activities of human class I HDAC by protein kinases [56]. MBD3 is one of the five members of the MBD family that recruits various HDAC containing repressor complexes leading to silencing by generating repressive chromatin structures at relevant binding sites. It plays an important role in mediating the HDAC-specific small-molecule inhibitors (HDI)-induced gene regulations associated with cancer-selective cell death, imparting HDI-induced selectivity in cancer cells via differential transcriptional regulation [59]. Silencing of MBD3 abrogates HDI-induced transcriptional reprogramming and growth inhibition in HDI-treated lung cancer cells but not in normal cells. In response to HDI treatment MBD3 relocalizes within cells in a different manner in cancer and normal cells, an indication that the relocation of MBD3 to the nucleus may facilitate its recruitment to the genome and allow MBD3 to function as a regulatory molecule [59]. Our ongoing studies have been designed for clarifying whether intracellular relocation plays a role in differential transcriptional reprogramming in response to pregnancy induced differentiation.

We found of great interest our findings that genes that are involved in the metabolism of xenobiotic substances and oxidative stress were significantly upregulated in the breast epithelium of postmenopausal parous women. Among them are the Epoxide hydrolase or EPHX1, which plays an important role in both the activation and detoxification of exogenous chemicals such as polycyclic aromatic hydrocarbons found in cigarette smoke [60], and thioredoxin reductase 1 or TXNRD [61], a member of the pyridine nucleotide family of oxidoreductases and one of the major antioxidant and redox regulators in mammals. TXNRD1 protein reduces thioredoxins and other substrates, playing a role in selenium metabolism, protecting against oxidative stress, and supporting the function of p53 and of other tumor suppressors. The upregulation in the parous breast epithelium of Glutathione S-transferase (GST) theta 1 (GSTT1), which belongs to a family of important enzymes involved in the detoxification of a wide variety of known and suspected carcinogens, including potential mammary carcinogens identified in charred meats and tobacco smoke is of importance because a substantial proportion of the Caucasian population has a homozygous deletion (null) of the GSTM1 or GSTT1 gene, which results in lack of production of these isoenzymes and a significantly elevated risk of breast cancer associated with cigarette smoking [62]. N-acetyltransferase 2-arylamine N-acetyltransferase (NAT2), that is involved in the metabolism of different xenobiotics, including potential carcinogens [63], indicates that the lifetime sequel of the differentiation of the breast by an early pregnancy is the activation of a system of defense that makes the parous breast cells less vulnerable to genotoxic substances.

This contention is supported by in vitro data demonstrating that breast epithelial cells from parous women do not express phenotypes of cell transformation when treated with chemical carcinogens, whereas those from nulliparous women do [64].

Seven DNA repair controlling genes were found to be significantly upregulated in the Lob 1 of the parous breast, an indication that an improved DNA repair system was involved in the protective effect induced by pregnancy, as we have previously demonstrated in the rodent experimental model in which mammary epithelial cells of parous animals remove 7-12 dimethylbenz (a) anthracene (DMBA) DNA adducts more efficiently than those of virgin animals [65]. DNA repair is central to the integrity of the human genome and reduced DNA repair capacity has been linked to genetic susceptibility to cancer, including that of the breast [66]. Among the genes that were upregulated in the epithelial cells of the parous breast were RAD51-like 3, or RAD51D, that is of the five RAD51 paralogs that are required in mammalian cells for normal levels of genetic recombination and damaging agents [67]. We have previously reported that the X ray repair complementing defective repair I (XRCC4) gene is up-regulated in breast epithelial cells of parous women[6,15,19,20]. XRCC4 is DNA repair factor that is essential for the resolution of DNA double strand break during V(D)J recombination, acting as a caretaker of the mammalian genome in both normal development and suppression of tumors. In the present study we found in the same cells the upregulation of Excision repair crosscomplementing rodent repair deficiency, complementation group 8 (ERCC8), also known as CSA [68], which interacts with CSB. And when mutated impair transcription-coupled repair (TCR), a DNA repair defect found in Cockayne syndrome [68]. The ankyrin repeat domain 17 or ANKRD17, the translin or TSN, that encodes a DNA-binding protein which specifically recognizes conserved target sequences at the breakpoint junction of chromosomal translocations [69], and the three prime repair exonuclease 1 (TREXI) are also upregulated in the parous control group. The protein encoded by this latter gene uses two different open reading frames from which the upstream ORF encodes proteins which interact with the ataxia telangiectasia and Rad3 related protein, a checkpoint kinase. The proteins encoded by this upstream ORF localize to intranuclear foci following DNA damage and are essential components of the DNA damage checkpoint [70,71]. These data indicate that the activation of genes involved in the DNA repair process is part of the signature induced in the mammary gland by pregnancy, confirming previous findings that in vivo, the ability of the cells to repair carcinogen-induced damage by unscheduled DNA synthesis and adduct removal is more efficient in the post pregnancy mammary gland [65].

Among the genes that control apoptosis eight were deregulated, six were up- and 2 downregulated. The former included the BCL2 associate X protein or BAX, a proapoptotic gene that belongs to the BCL2 protein family whose transcription is stimulated by the active p53 and the pro-apoptotic and cell cycle regulator gene p21 [72]. To the same category belongs the cytotoxic granule-associated RNA binding protein (TIA1), tumor necrosis factor TNF receptorassociated factor 1 (TRAF1), TRADD, CASP2 and RIPK1 domain containing adaptor with death domain or CRADD, and Protein phosphatase 1F (PPM1F). TIA1 possesses nucleolytic activity against cytotoxic lymphocyte (CTL) target cells inducing in them DNA fragmentation [73].

TNFR1 can initiate several cellular responses, including apoptosis that relies on caspases, and necrotic cell death, which depends on receptor-interacting protein kinase 1 (RIP1) [74,75]. TRADD protein has been suggested to be a crucial signal adaptor that mediates all intracellular responses from TNFR1 [76]. Caspase-2 is one of the earliest identified caspases engaged in the mitochondria-dependent apoptotic pathway by inducing the release of cytochrome c (Cyt c) and other mitochondrial apoptogenic factors into the cell cytoplasm [77]. PPMIF encodes a protein that is a member of the PP2C family of Ser/Thr protein phosphatases; overexpression of this phosphatase has been shown to mediate caspase-dependent apoptosis [78]. Two apoptotic and two antiapoptotic genes are downregulated in the breast epithelium of parous women: the programmed cell death 5 or PDCD5- and the transformed 3T3 cell double minute 4 (MDM4) in the former and Baculoviral inhibition of apoptosis protein (IAP) repeat-containing 6 (BIRC6) and BCL2-associated athanogene 4 (BAG4) in the latter. The Mdm4 gene that encodes structurally related oncoproteins that bind to the p53 tumor suppressor protein and inhibit p53 activity is amplified and overexpressed in a variety of human cancers [79]. The Split hand/foot malformation (ectrodactyly) type 1-encodes a protein with a BIR (baculoviral) domain and UBCc (ubiquitin-conjugating enzyme E2, catalytic) domain [80]. This protein inhibits apoptosis by facilitating the degradation of apoptotic proteins by ubiquitination. BAG4 is a member of the BAG1-related anti-apoptotic protein family that functions through interactions with a variety of cell apoptosis and growth related proteins including BCL-2, Raf-protein kinase, steroid hormone receptors, growth factor receptors, and members of the heat shock protein 70 kDa family. This protein was found to be associated with the death domain of tumor necrosis factor receptor type 1 (TNF-R1) and death receptor-3 (DR3), and thereby negatively regulates downstream cell death signaling [81]. Altogether these clusters of genes seem to maintain the programmed cell death pathway very active in the parous breast epithelium when compared with the epithelium obtained from the breast of parous women with cancer and from nulliparous women with or without cancer. Supporting evidence for this statement comes from data obtained from experimental models [6,21,22] and from reduction mammoplasty normal breast tissue of parous women [25-27], in which genes involved in the pathway of apoptosis are significantly deregulated. Another cluster of genes that are upregulated in the parous control group are those related to immunosurveillance. We have previously reported that breast epithelial cells from parous women significantly overexpressed genes related to the immune system [82], therefore this category will not be further discussed here.

A preferred genetic signature comprises the nucleic acids encoding the protein products listed in Table 4.

| | | | | Molecular |
|---|---|---|---|---|
| | | | | Function |
| | | | | GO |
| Gene name | Gene ID | Symbol | GO number | number |
| BCL2-associated X protein | AI565203 | BAX | GO: 0006915 | GO: 0005515 |
| Epoxide hydrolase 1, microsomal (xenobiotic) | AA838691 | EPHX1 | GO: 0006805 | GO: 0004301 |
| Excision repair cross-complementing rodent | N49276 | ERCC8 | GO: 0006281 | GO: 0003702 |
| repair deficiency | | | | |
| RAD51-like 3 (S. cerevisiae) | N29765 | RAD51L3 | GO: 0006284 | GO: 0005524 |
| Homeobox D1 | W68537 | HOXD1 | GO: 0006355 | GO: 0003700 |
| p300/CBP-associated factor | N74637 | PCAF | GO: 0006350 | 0.001368735 |
| Inhibitor of DNA binding 4 | AA464856 | ID4 | GO: 0006357 | GO: 0003714 |
| Suppressor of Ty 5 homolog (S. cerevisiae) | R21511 | SUPT5H | GO: 0000122 | GO: 0003711 |
| Histone deacetylase 8 | AI053481 | HDAC8 | GO: 0000122 | GO: 0004407 |
| Methyl-CpG binding domain protein 3 | AI017865 | MBD3 | GO: 0006350 | GO: 0003677 |

Altogether our data indicate that the first full term pregnancy induces in the breast epithelium a specific genomic profile that is still identifiable in parous women at postmenopause. Furthermore, this genomic signature is constituted by genes that cluster differently than those genes expressed in the epithelial cells of parous and nulliparous women with breast cancer as well as from nulliparous women without cancer. This genomic signature allowed us to evaluate the degree of mammary gland differentiation induced by pregnancy and could become the signature postulated for the Stem Cell 2 (31, 66-68). Of importance is the fact that this signature serves for characterizing at molecular level the fully differentiated condition of the breast epithelium that is associated with a reduction in breast cancer risk, thus providing a useful molecular tool for predicting when pregnancy has been protective, for identifying women at risk irrespective of their pregnancy history, and for its use as an intermediate biomarker for evaluating cancer preventive agents.

### REFERENCES

1. Mustacchi P. Ramazzini and Rigoni-Stern on parity and breast cancer. Clinical impression and statistical corroboration. Arch Intern Med 1961; 108:639-42.
2. MacMahon B, Cole P, Lin T M. Age at first birth and breast cancer risk. Bull World Health Organ 1970; 43: 209-21.
3. Tryggvadottir L, Tulinius H, Eyfjord J E, Sigurvinsson T. Breastfeeding and reduced risk of breast cancer in an Icelandic cohort study. Am J Epidemiol 2001; 154: 37-42.
4. Jernstrom H, Lubinski J, Lynch H T, et al. Breast-feeding and the risk of breast cancer in BRCA1 and BRCA2 mutation carriers. J Natl Cancer Inst 2004; 96:1094-98.
5. Russo J, Tay L K, Russo I H. Differentiation of the mammary gland and susceptibility to carcinogenesis: A Review. Breast Cancer Res Treat 1982; 2:5-73.
6. Russo J, Balogh G A, Chen J, et al. The concept of stem cell in the mammary gland and its implication in morphogenesis, cancer and prevention. Frontiers in Bioscience 2006; 11:151-72.
7. Cutuli B, Borel C, Dhermain F, et al. Breast cancer occurred after treatment for Hodgkin's disease: analysis of 133 cases. Radiother Oncol 2001; 59:247-55.
8. Key J, Hodgson S, Omar R Z, et al. Meta-analysis of studies of alcohol and breast cancer with consideration of the methodological issues. Cancer Causes Control 2006; 17:759-70.
9. Band P R, Le N D, Fang R, Deschamps M. Carcinogenic and endocrine disrupting effects of cigarette smoke and risk of breast cancer. Lancet 2002; 360:1044-49.
10. Russo J, Tait L, Russo I H. Susceptibility of the mammary gland to carcinogenesis: III the cell of origin of rat mammary carcinoma. Am J Path 1983; 113:50-66.
11. Shantakumar S, Terry M B, Teitelbaum S L, et al. Reproductive factors and breast cancer risk among older women. Breast Cancer Res Treat 2007; 102:365-74.
12. Iwasaki M, Otani T, Inoue M, et al. Role and impact of menstrual and reproductive factors on breast cancer risk in Japan. Eur J Cancer Prey 2007; 16:116-23.
13. Ha M, Mabuchi K, Sigurdson A J, et al. Smoking Cigarettes before First Childbirth and Risk of Breast Cancer. Am J Epidemiol 2007; 166:55-61.
14. Kote-Jarai Z, Matthews L, Osorio A, et al. Accurate prediction of BRCA1 and BRCA2 heterozygous genotype using expression profiling after induced DNA damage. Clin Cancer Res 2006; 12:3896-901.
15. Epidemiological Study of BRCA1 and BRCA2 Mutation Carriers (EMBRACE); Gene Etude Prospective Sein Ovaire (GENEPSO); Gen en Omgeving studie van de werkgroep Hereditiair Borstkanker Onderzoek Nederland (GEO-HEBON); International BRCA1/2 Carrier Cohort Study (IBCCS) Collaborators' Group; Andrieu N, Easton D F, Chang-Claude J, et al. Effect of chest X-rays on the risk of breast cancer among BRCA1/2 mutation carriers in the international BRCA1/2 carrier cohort study: a report from the EMBRACE, GENEPSO, GEO-HEBON, and IBCCS Collaborators' Group. J Clin Oncol 2006; 24:3361-66.
16. Russo J, Lynch H, Russo I H. Mammary gland architecture as a determining factor in the susceptibility of the human breast to cancer. Breast J 2001; 7:278-91.
17. Russo J, Russo I H. Differentiation and breast cancer development. In: Heppner G. (Ed) Advances in oncobiology. Vol. 2. JAI Press Inc. 1998. p. 1-10.
18. Russo J, Russo I H. Toward a Unified Concept of Mammary Tumorigenesis. Prog Clin Biol Res 1997; 396:1-16.
19. Russo J, Ao X, Grill C, Russo I H. Pattern of distribution of cells positive for estrogen receptor a and progesterone receptor in relation to proliferating cells in the mammary gland. Breast Cancer Res Treat 1999; 53:217-27.
20. Russo J, Gusterson B A, Rogers A E, et al. Comparative study of human and rat mammary tumorigenesis. Lab Invest 1990; 62:244-78.
21. Russo J, Mailo D, Hu Y-F et al. Breast differentiation and its implication in cancer prevention. Clin Cancer Res 2005; 11:931s-6s.
22. Russo I H, Russo J. Developmental stage of the rat mammary gland as determinant of its susceptibility to 7,12-dimethylbenz(a)anthracene. J Natl Cancer Inst 1978; 61:1439-49.
23. Grubbs C J, Hill D L, McDonough K C, Peckham J C. N-nitroso-N-methylurea-induced mammary carcinogenesis: effect of pregnancy on preneoplastic cells. J Natl Cancer Inst 1983; 71:625-8.
24. Blakely C M, Stoddard A J, Belka G K et al. Hormone-induced protection against mammary tumorigenesis is conserved in multiple rat strains and identifies a core gene expression signature induced by pregnancy. Cancer Res 2006; 66, 6421-31.
25. Balogh G A, Heulings R, Mailo D A, et al. Genomic signature induced by pregnancy in the human breast. Int J Oncol 2006; 28:399-410.
26. Russo J, Balogh G A, Heulings R, et al. Molecular basis of pregnancy induced breast cancer protection. Eur J Cancer Prey 2006; 15:306-42.
27. Balogh G A, Heulings R, Mailo D, Wang R, Li Y S, Hardy R, Russo J. Methodological approach to study the genomic profile of the human breast. Int J. Oncol. 2007; 31:253-60.
28. Gentleman R C, Carey V J, Bates D M, et al. Bioconductor: open software development for computational biology and bioinformatics. Genome Biol 2004; 5:R80.
29. Wettenhall J M, Smyth G K. LimmaGUI: A graphical user interface for linear modeling of microarray data. Bioinformatics 2004; 20:3705-6.
30. Smyth G K. Limma: linear models for microarray data. In: Gentleman R, Carey V, Dudoit S, Irizarry R, Huber W, editors. Bioinformatics and Computational Biology Solutions using R and Bioconductor. New York: Springer; 2005. p 397-420.
31. Benjamini Y, Hochberg Y. Controlling the false discovery rate: a practical and powerful approach to multiple testing. J Royal Stat Soc B 1995; 57:289-300.
32. Ritchie M E, Diyagama D, Neilson J, et al. Empirical array quality weights for microarray data. BMC Bioinformatics 2006; 7, 261.
33. Perelman E, Ploner A, Calza S, Pawitan Y. Detecting differential expression in microarray data: comparison of optimal procedures. BMC Bioinformatics. 2007; 8:28.
34. Maglietta R, Piepoli A, Catalano D, et al. Statistical assessment of functional categories of genes deregulated in pathological conditions by using microarray data. Bioinformatics. 2007; May 31 [Epub ahead of print].
35. Stachora A A, Schafer R E, Pohlmeier M, Maier G, Ponstingl H. Human Supt5h protein, a putative modulator of chromatin structure, is reversibly phosphorylated in mitosis. FEBS Lett 1997; 409: 74-8.
36. Stasinopoulos I, O'Brien D R, Wildes F, Glunde K, Bhujwalla Z M. Silencing of cyclooxygenase-2 inhibits metastasis and delays tumor onset of poorly differentiated metastatic breast cancer cells. Mol Cancer Res 2007; 5:435-42.
37. Kurochkin I V, Yonemitsu N, Funahashi S I, Nomura H. ALEX1, a novel human armadillo repeat protein that is expressed differentially in normal tissues and carcinomas. Biochem Biophys Res Commun 2001; 280:340-7.
38. Hsia N, Cornwall G A. DNA microarray analysis of region-specific gene expression in the mouse epididymis. Biol Reprod 2004; 70:448-57.
39. Roldan G, Delgado L, Muse I M. Tumoral expression of BRCA1, estrogen receptor alpha and ID4 protein in patients with sporadic breast cancer. Cancer Biol Ther 2006; 5:505-10.
40. de Candia P, Akram M, Benezra R, Brogi E. Id4 messenger RNA and estrogen receptor expression: inverse correlation in human normal breast epithelium and carcinoma. Hum Pathol 2006; 37:1032-41.
41. Yu L, Liu C, Vandeusen J, et al. Global assessment of promoter methylation in a mouse model of cancer identifies ID4 as a putative tumor-suppressor gene in human leukemia. Nat Genet. 2005; 37:265-74.
42. Umetani N, Takeuchi H, Fujimoto A, et al. Epigenetic inactivation of ID4 in colorectal carcinomas correlates with poor differentiation and unfavorable prognosis. Clin Cancer Res 2004; 10:7475-83.
43. Chan A S, Tsui W Y, Chen X, et al. Downregulation of ID4 by promoter hypermethylation in gastric adenocarcinoma. Oncogene 2003; 22:6946-53.
44. Umetani N, Mori T, Koyanagi K. Aberrant hypermethylation of ID4 gene promoter region increases risk of lymph node metastasis in T1 breast cancer. Oncogene 2005; 24:4721-7.
45. Kim J Y, Tavaré S, Shibata D. Human hair genealogies and stem cell latency. BMC Biol 2006; 4:2.
46. Ito Y, Wiese S, Funk N, et al. Sox10 regulates ciliary neurotrophic factor gene expression in Schwann cells. Proc Natl Acad Sci USA 2006; 103:7871-6.
47. Weiss J, Meeks J J, Hurley L, Raverot G, Frassetto A, Jameson J L. Sox3 is required for gonadal function, but not sex determination, in males and females. Mol Cell Biol 2003; 23:8084-91.
48. Raverot G, Weiss J, Park S, Hurley L, Jameson J L. Sox3 expression in undifferentiated spermatogonia is required for the progression of spermatogenesis. Dev Biol 2005; 283:215-25.
49. Dutertre M, Smith C L. Ligand-Independent Interactions of p160/Steroid Receptor Coactivators and CREB-Binding Protein (CBP) with Estrogen Receptor-a: Regulation by Phosphorylation Sites in the A/B Region Depends on Other Receptor Domains Mol. Endocrinol. 2003; 17: 1296-314.
50. Iwase H. Molecular action of the estrogen receptor and hormone dependency in breast cancer. Breast Cancer 2003; 10:89-96.
51. Watts G S, Oshiro M M, Junk D J, et al. The acetyltransferase p300/CBP-associated factor is a p53 target gene in breast tumor cells. Neoplasia 2004; 6:187-94.
52. Hayashi K, Goodison S, Urquidi V, et al. Differential effects of retinoic acid on the growth of isogenic metastatic and non-metastatic breast cancer cell lines and their association with distinct expression of retinoic acid receptor beta isoforms 2 and 4. Int J Oncol 2003; 22:623-9.
53. Manohar C F, Salwen H R, Furtado M R, Cohn S L. Up-regulation of HOXC6, HOXD1, and HOXD8 homeobox gene expression in human neuroblastoma cells following chemical induction of differentiation. Tumour Biol 1996; 17:34-47.
54. Khwaja A. The role of Janus kinases in haemopoiesis and haematological malignancy. Brit J Haematol 2006; 134:366-84.
55. Clevenger C V. Roles and Regulation of Stat Family Transcription Factors in Human Breast Cancer. Am J Pathol 2004; 165: 1449-60.
56. Lee H, Rezai-Zadeh N, Seto E. Negative Regulation of Histone Deacetylase 8 Activity by Cyclic AMP-Dependent Protein Kinase A. Mol Cell Biol 2004; 24:765-73.
57. Melnick A, Licht J D. Histone deacetylases as therapeutic targets in hematologic malignancies. Curr Opin Hematol 2002; 9:322-32.
58. Jiang X, Russo I H, Russo J. Human chorionic gonadotropin and inhibin induce histone acetylation in human breast cancer cells. Int J Oncol 2002; 20:77-9.
59. Noh E J, Jong E R, Jeong G, Lee Y M, Min C K, Lee J-S. Methyl CpG-binding domain protein 3 mediates cancer-selective cytotoxicity by histone deacetylase inhibitors via differential transcriptional reprogramming in lung cancer cells. Cancer Res 2005; 65:11400-10.
60. Huang W-Y, Chatterjee N, Chanock S, et al. Microsomal epoxide hydrolase polymorphisms and risk for advanced colorectal adenoma. Cancer Epidemiol Biomarkers Prey 2005; 14:152-7.
61. Yoo M H, Xu X M, Carlson B A, Gladyshev V N, Hatfield D L. Thioredoxin reductase 1 deficiency reverses tumor phenotype and tumorigenicity of lung carcinoma cells. J Biol Chem 2006; 281:13005-8.
62. Zheng W, Wen W Q, Gustafson D R, Gross M, Cerhan J R, Folsom A R. GSTM1 and GSTT1 polymorphisms and postmenopausal breast cancer risk. Breast Cancer Res Treat 2002; 74:9-16.
63. Mucci L A, Wedren S, Tamimi R M, Trichopoulos D, Adami H O. The role of gene environment interaction in the aetiology of human cancer: examples from cancers of the large bowel, lung and breast. J Int Med 2001; 249:477-93.
64. Russo J, Calaf G, Sohi N, et al Critical steps in breast carcinogenesis. Ann N Y Acad Sci 1993; 698:1-20.
65. Tay L K, Russo J. Formation and removal of 7,12-dimethylbenz(a)-anthracenenucleic acid adducts in rat mammary epithelial cells with different susceptibility to carcinogenesis. Carcinog 1981; 2:1327-33.
66. Kennedy D O, Agrawal M, Shen J, et al. DNA repair capacity of lymphoblastoid cell lines from sisters discordant for breast cancer. J Natl Cancer Inst 2005; 97:127-32.
67. Wiese C, Hinz J M, Tebbs R S, et al. Disparate requirements for the Walker A and B ATPase motifs of human RAD51D in homologous recombination. Nucleic Acid Res 2006; 34:2833-43.
68. Laine J P, Egly J M. When transcription and repair meet: a complex system. Trends Genet 2006; 22:430-436.
69. Mellon S H, Bair S R, Depoix C, Vigne J L, Hecht N B, Brake P. Translin coactivates steroidogenic factor-1-stimulated transcription. Mol Endocrinol 2007; 21:89-105.
70. Bomgarden R D, Yean D, Yee M C, Cimprich K A. A novel protein activity mediates DNA binding of an ATR-ATRIP complex. J Biol Chem 2004; 279:13346-53.
71. Zou L, Elledge S J. Sensing DNA damage through ATRIP recognition of RPAssDNA complexes. Science 2003; 300:1542-48.
72. Eissing T, Waldherr S, Allgower F, Scheurich P, Bullinger E. Response to bistability in apoptosis: roles of bax, bcl-2, and mitochondrial permeability transition pores. Biophys J 2007; 92:3332-4.
73. Mori N, Murakami Y I, Shimada S, et al. TIA-1 expression in hairy cell leukemia. Mod Pathol 2004; 17:840-6.
74. Xie P, Hostager B S, Munroe M E, Moore C R, Bishop G A. Cooperation between TNF receptor-associated factors 1 and 2 in CD40 signaling. J Immunol 2006; 176:5388-400.
75. Bryce P J, Oyoshi M K, Kawamoto S, Oettgen H C, Tsitsikov E N. TRAF1 regulates Th2 differentiation, allergic inflammation and nuclear localization of the Th2 transcription factor, NIP45. Int Immunol 2006; 18:101-11.
76. Zheng L, Bidere N, Staudt D, et al. Competitive control of independent programs of tumor necrosis factor receptor-induced cell death by TRADD and RIP1. Mol Cell Biol 2006; 26:3505-13.
77. Guo Y, Srinivasula S M, Druilhe A, Fernandes-Alnemri T, Alnemri E S. Caspase-2 induces apoptosis by releasing proapoptotic proteins from mitochondria. J Biol Chem 2002; 277:13430-7.
78. Mi J, Guo C, Brautigan D L, Larner J M. Protein Phosphatase-1 { alpha} Regulates Centrosome Splitting through Nek2. Cancer Res 2007; 67:1082-9.
79. Francoz S, Froment P, Bogaerts S, et al. (Mdm4 and Mdm2 cooperate to inhibit p53 activity in proliferating and quiescent cells in vivo. Proc Natl Acad Sci USA. 2006; 103:3232-7.
80. Colnaghi R, Connell C M, Barrett R M, Wheatley S P. Separating the anti-apoptotic and mitotic roles of survivin. J Biol Chem 2006; 281:33450-6.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A genetic signature of differentially expressed nucleic acids, said differential expression being associated with a reduced risk of breast cancer conferred by full term pregnancy, said signature comprising at least 4, 5 or 10 of the differentially expressed nucleic acids provided in Tables 2 or 3.

2. A genetic signature of differentially expressed nucleic acids, said differential expression being associated with a reduced risk of breast cancer conferred by full term pregnancy, said signature comprising at least 4, 5 or all of the differentially expressed nucleic acids provided in Table 4.

3. The genetic signature of claim 1 or 2 obtained by comparing hybridization profiles from cells of
i) parous women with breast cancer;
ii) nulliparous women with breast cancer;
iii) parous women without breast cancer
iv) nulliparous women without breast cancer, said genetic signature associated with a reduced breast cancer risk, and comprising those particular nucleic acid sequences which are differentially expressed between said parous and nulliparous women with and without breast cancer.

4. A plurality of protein products encoded by the nucleic acids set forth in Table 2, 3 or 4.

5. A method for identifying agents which alter the activity of at least one protein product encoded by the nucleic acids present in the genetic signature of claim 1 or 2, comprising;
a) contacting breast cells from parous and nulliparous women with said agent;
b) assessing said cells for a parameter associated with malignant transformation, agents which alter said parameter being effective to alter the activity of said at least one breast cancer genetic signature biomarker.

6. The method of claim 6, wherein said parameter is selected from the group consisting of altered cellular proliferation rate, altered apoptosis, altered cellular morphology, and altered cellular viability.

7. An agent identified by the method of claim 5.

8. An agent as claimed in claim 7, wherein said agent is selected from the group consisting of a small molecule, an antibody, a protein, an oligonucleotide, or an siRNA molecule.

9. A method for diagnosing a reduced risk for the development of breast cancer in a patient comprising;
a) obtaining a sample of breast cells from said patient;
b) determining differential expression levels of nucleic acids isolated from said cells thereby obtaining a genetic signature from said patient; and
c) comparing the genetic signature from said patient to the genetic signatures of claim 1 or 2, wherein when said signatures are comparable, said patient has a reduced risk for developing breast cancer.

10. A kit for practicing the method of claim 8, said kit comprising a microarray comprising at least the nucleic acids provided in Table 2, means for isolating nucleic acids from breast cells, means for incorporating detectable labels into said isolated nucleic acids, and reagents suitable for conducting a hybridization reaction.
